SEQUENCE LISTING (38 sequences)

SEQ ID NO: 1 - DNA, 3258 bp, artificial
  SV2-SNAP25-tTA (2)(pcDNA3.1)
gctagcgccaccatggaagactcttacaaggataggacttcactgatgaagggtgccaaggacattgccagagaggtgaagaaacaaacagtaaagaaggtgaatcaagctgtggaccgagcccaggatgaatacacccagaggtcctacagtcggttccaagatgaagaagatgatgatgactactacccggctggagaaacctataatggtgaggccaacgatgacgaaggctcaagtgaagccactgaggggcatgatgaagatgatgagatctatgagggggagtatcagggcatccccagtatgaaccaagcgaaggacagcatcgtgtcagtggggcagcccaagggcgatgagtacaaggaccgacgggagctggaatcagaaaggagagctgacgaggaagagttagcccagcagtatgagctgataatccaagaatgcggtcatggtcgttttcagtgggcccttttcttcgtcctgggcatggctcttatggcagacggtgtagaggtgtttgtcgttggcttcgtgttacccagtgctgagacagacctctgcatcccaaattcaggatctggatggctaggcagcatagtgtacctcgggatgatggtgggggcgttcttctggggaggactggcagacaaagtgggaaggaaacagtctcttctgatttgcatgtctgtcaacggattctttgccttcctttcttcatttgtccaaggttatggcttctttctcttctgtcgcttactttctggattcgggattggaggagccatacccactgtgttctcgtactttgctgaagtcctggcccgggaaaagcggggcgaacacttgagctggctctgcatgttctggatgatcggtggcatctacgcctctgccatggcctgggccatcatcccgcactacgggtggagcttcagcatgggatcggcctaccagtttcacagttggcgtgtgtttgtcatcgtctgtgcactcccctgtgtctcctccgtggtggccctcacattcatgcctgaaagcccacgattcttgttggaggttggaaaacatgatgaagcttggatgattctgaagttaattcatgacaccaacatgagagcccggggtcagcctgagaaggtcttcacggtaaacaaaataaaaactcctaaacaaatagatgagctgattgaaattgagagtgacacaggaacatggtataggaggtgttttgttcggatccgcaccgagctgtacggaatttggttgacttttatgagatgtttcaactacccagtcagggataatacaataaagcttacaattgtttggttcaccctgtcctttgggtactatggattatccgtttggttccctgatgtcattaaacctctgcagtccgatgaatatgcattgctaaccagaaatgtggagagagataaatatgcaaatttcactattaactttacaatggaaaatcagattcatactggaatggaatacgacaatggcagattcataggggtcaagttcaaatctgtaactttcaaagactctgtttttaagtcctgcacctttgaggatgtaacttcagtgaacacctacttcaagaactgcacatttattgacactgtttttgacaacacagattttgagccatataaattcattgacagtgaatttaaaaactgctcgttttttcacaacaagacgggatgtcagattacctttgatgatgactatagtgcctactggatttattttgtcaactttctggggacattggcagtattgccagggaacattgtgtctgctctgctgatggacagaattgggcgcttaacaatgctaggtggctctatggtgctttcggggatcagctgtttcttcctttggttcggcaccagtgaatccatgatgataggcatgctgtgtctgtacaatggattgaccatctcagcctggaactctcttgacgtggtcactgtggaactgtaccccacagaccggagggcaacaggctttggcttcttaaatgcgctatgcaaggcagcagccgtcctgggaaacttaatatttggctctctggtcagcatcaccaaatcaatccccatcctgctggcttctactgtgctcgtgtgtggaggactcgttgggctgtgcctgcctgacacacgatcaagtgatgcttacaaaaaagcctggggcaataatcaggacggagtggtggccagccagcctgctcgtgtagtggacgaacgggagcagatggccatcagtggcggcttcatccgcagggtaacaaatgatgcccgagaaaatgaaatggatgaaaacctagagcaggtgagcggcatcatcgggaacctccgtcacatggccctggatatgggcaatgagatcgatacacagaatcgccagatcgacaggatcatggagaaggctgattccaacaaaaccagaattgatgaggccaaccaacgtgcaacaaagatgctgggaagtggttctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaactcgag

SEQ ID NO: 2 - protein, 1079 aa, artificial
  SV2-SNAP25-tTA (2)(pcDNA3.1)
MEDSYKDRTSLMKGAKDIAREVKKQTVKKVNQAVDRAQDEYTQRSYSRFQDEEDDDDYYPAGETYNGEANDDEGSSEATEGHDEDDEIYEGEYQGIPSMNQAKDSIVSVGQPKGDEYKDRRELESERRADEEELAQQYELIIQECGHGRFQWALFFVLGMALMADGVEVFVVGFVLPSAETDLCIPNSGSGWLGSIVYLGMMVGAFFWGGLADKVGRKQSLLICMSVNGFFAFLSSFVQGYGFFLFCRLLSGFGIGGAIPTVFSYFAEVLAREKRGEHLSWLCMFWMIGGIYASAMAWAIIPHYGWSFSMGSAYQFHSWRVFVIVCALPCVSSVVALTFMPESPRFLLEVGKHDEAWMILKLIHDTNMRARGQPEKVFTVNKIKTPKQIDELIEIESDTGTWYRRCFVRIRTELYGIWLTFMRCFNYPVRDNTIKLTIVWFTLSFGYYGLSVWFPDVIKPLQSDEYALLTRNVERDKYANFTINFTMENQIHTGMEYDNGRFIGVKFKSVTFKDSVFKSCTFEDVTSVNTYFKNCTFIDTVFDNTDFEPYKFIDSEFKNCSFFHNKTGCQITFDDDYSAYWIYFVNFLGTLAVLPGNIVSALLMDRIGRLTMLGGSMVLSGISCFFLWFGTSESMMIGMLCLYNGLTISAWNSLDVVTVELYPTDRRATGFGFLNALCKAAAVLGNLIFGSLVSITKSIPILLASTVLVCGGLVGLCLPDTRSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSGSRLDKSKVINSALELLNGVGIEGLTTRKLAQKLGVEQPTLYWHVKNKRALLDALPIEMLDRHHTHSCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 3 - DNA, 2814 bp, artificial
  SV2-SNAP25-tTA (3)(pCMV-Tet3G)
gaattcaccatggaagactcttacaaggataggacttcactgatgaagggtgccaaggacattgccagagaggtgaagaaacaaacagtaaagaaggtgaatcaagctgtggaccgagcccaggatgaatacacccagaggtcctacagtcggttccaagatgaagaagatgatgatgactactacccggctggagaaacctataatggtgaggccaacgatgacgaaggctcaagtgaagccactgaggggcatgatgaagatgatgagatctatgagggggagtatcagggcatccccagtatgaaccaagcgaaggacagcatcgtgtcagtggggcagcccaagggcgatgagtacaaggaccgacgggagctggaatcagaaaggagagctgacgaggaagagttagcccagcagtatgagctgataatccaagaatgcggtcatggtcgttttcagtgggcccttttcttcgtcctgggcatggctcttatggcagacggtgtagaggtgtttgtcgttggcttcgtgttacccagtgctgagacagacctctgcatcccaaattcaggatctggatggctaggcagcatagtgtacctcgggatgatggtgggggcgttcttctggggaggactggcagacaaagtgggaaggaaacagtctcttctgatttgcatgtctgtcaacggattctttgccttcctttcttcatttgtccaaggttatggcttctttctcttctgtcgcttactttctggattcgggattggaggagccatacccactgtgttctcgtactttgctgaagtcctggcccgggaaaagcggggcgaacacttgagctggctctgcatgttctggatgatcggtggcatctacgcctctgccatggcctgggccatcatcccgcactacgggtggagcttcagcatgggatcggcctaccagtttcacagttggcgtgtgtttgtcatcgtctgtgcactcccctgtgtctcctccgtggtggccctcacattcatgcctgaaagcccacgattcttgttggaggttggaaaacatgatgaagcttggatgattctgaagttaattcatgacaccaacatgagagcccggggtcagcctgagaaggtcttcacggtaaacaaaataaaaactcctaaacaaatagatgagctgattgaaattgagagtgacacaggaacatggtataggaggtgttttgttcggatccgcaccgagctgtacggaatttggttgacttttatgagatgtttcaactacccagtcagggataatacaataaagcttacaattgtttggttcaccctgtcctttgggtactatggattatccgtttggttccctgatgtcattaaacctctgcagtccgatgaatatgcattgctaaccagaaatgtggagagagataaatatgcaaatttcactattaactttacaatggaaaatcagattcatactggaatggaatacgacaatggcagattcataggggtcaagttcaaatctgtaactttcaaagactctgtttttaagtcctgcacctttgaggatgtaacttcagtgaacacctacttcaagaactgcacatttattgacactgtttttgacaacacagattttgagccatataaattcattgacagtgaatttaaaaactgctcgttttttcacaacaagacgggatgtcagattacctttgatgatgactatagtgcctactggatttattttgtcaactttctggggacattggcagtattgccagggaacattgtgtctgctctgctgatggacagaattgggcgcttaacaatgctaggtggctctatggtgctttcggggatcagctgtttcttcctttggttcggcaccagtgaatccatgatgataggcatgctgtgtctgtacaatggattgaccatctcagcctggaactctcttgacgtggtcactgtggaactgtaccccacagaccggagggcaacaggctttggcttcttaaatgcgctatgcaaggcagcagccgtcctgggaaacttaatatttggctctctggtcagcatcaccaaatcaatccccatcctgctggcttctactgtgctcgtgtgtggaggactcgttgggctgtgcctgcctgacacacgaacccaggttctgatggccgaagacgcagacatgcgcaatgagctggaggagatgcagcgaagggctgaccagttggctgatgagtcgctggaaagcacccgtcgtatgctgcaactggttgaagagagtaaagatgctggtatcaggactttggttatgttggatgaacaaggagaacaactggaacgcattgaggaagggatggaccaaatcaataaggacatgaaagaagcagaaaagaatttgacggacctaggaaaattctgcgggctttgtgtgtgtccctgtaacaagcttaaatcaagtgatgcttacaaaaaagcctggggcaataatcaggacggagtggtggccagccagcctgctcgtgtagtggacgaacgggagcagatggccatcagtggcggcttcatccgcagggtaacaaatgatgcccgagaaaatgaaatggatgaaaacctagagcaggtgagcggcatcatcgggaacctccgtcacatggccctggatatgggcaatgagatcgatacacagaatcgccagatcgacaggatcatggagaaggctgattccaacaaaaccagaattgatgaggccaaccaacgtgcaacaaagatgctgggaagtggttaatctaga

SEQ ID NO: 4 - protein, 1179 aa, artificial [sequence omitted]

SEQ ID NO: 5 - DNA, 1725 bp, artificial
  Cholinetransporter-SNAP25-tTA (1)(pCMV-Tet3G)
gaattcaccatggctttccatgtggaaggactgatagctatcatcgtgttctaccttctaattttgctggttggaatatgggctgcctggagaaccaaaaacagtggcagcgcagaagagcgcagcgaagccatcatagttggtggccgagatattggtttattggttggtggatttaccatgacagctacctgggtcggaggagggtatatcaatggcacagctgaagcagtttatgtaccaggttatggcctagcttgggctcaggcaccaattggatattctcttagtctgattttaggtggcctgttctttgcaaaacctatgcgttcaaaggggtatgtgaccatgttagacccgtttcagcaaatctatggaaaacgcatgggcggactcctgtttattcctgcactgatgggagaaatgttctgggctgcagcaattttctctgctttgggagccaccatcagcgtgatcatcgatgtggatatgcacatttctgtcatcatctctgcactcattgccactctgtacacactggtgggagggctctattctgtggcctacactgatgtcgttcagctcttttgcatttttgtagggctgtggatcagcgtcccctttgcattgtcacatcctgcagtcgcagacatcgggttcactgctgtgcatgccaaataccaaaagccgtggctgggaactgttgactcatctgaagtctactcttggcttgatagttttctgttgttgatgctgggtggaatcccatggcaagcatactttcagagggttctctcttcttcctcagccacctatgctcaagtgctgtccttcctggcagctttcgggtgcctggtgatggccatcccagccatactcattggggccattggagcatcaacagactggaaccagactgcatatgggcttccagatcccaagactacagaagaggcagacatgattttaccaattgttctgcagtatctctgccctgtgtatatttctttctttggtcttggtgcagtttctgctgctgttatgtcatcagcagattcttccatcttgtcagcaagttccatgtttgcacggaacatctaccagctttccttcagacaaaatgcttcggacaaagaaatcgtttgggttatgcgaatcacagtgtttgtgtttggagcatctgcaacagccatggccttgctgacgaaaactgtgtatgggctctggtacctcagttctgaccttgtttacatcgttatcttcccccagctgctttgtgtactctttgttaagggaaccaacacctatggggccgtggcaggttatgtttctggcctcttcctgagaataactggaggggagccatatctgtatcttaacaagcttaaatcaagtgatgcttacaaaaaagcctggggcaataatcaggacggagtggtggccagccagcctgctcgtgtagtggacgaacgggagcagatggccatcagtggcggcttcatccgcagggtaacaaatgatgcccgagaaaatgaaatggatgaaaacctagagcaggtgagcggcatcatcgggaacctccgtcacatggccctggatatgggcaatgagatcgatacacagaatcgccagatcgacaggatcatggagaaggctgattccaacaaaaccagaattgatgaggccaaccaacgtgcaacaaagatgctgggaagtggttctaga

SEQ ID NO: 6 - protein, 817 aa, artificial
  Cholinetransporter-SNAP25-tTA (1)(pCMV-Tet3G)
MAFHVEGLIAIIVFYLLILLVGIWAAWRTKNSGSAEERSEAIIVGGRDIGLLVGGFTMTATWVGGGYINGTAEAVYVPGYGLAWAQAPIGYSLSLILGGLFFAKPMRSKGYVTMLDPFQQIYGKRMGGLLFIPALMGEMFWAAAIFSALGATISVIIDVDMHISVIISALIATLYTLVGGLYSVAYTDVVQLFCIFVGLWISVPFALSHPAVADIGFTAVHAKYQKPWLGTVDSSEVYSWLDSFLLLMLGGIPWQAYFQRVLSSSSATYAQVLSFLAAFGCLVMAIPAILIGAIGASTDWNQTAYGLPDPKTTEEADMILPIVLQYLCPVYISFFGLGAVSAAVMSSADSSILSASSMFARNIYQLSFRQNASDKEIVWVMRITVFVFGASATAMALLTKTVYGLWYLSSDLVYIVIFPQLLCVLFVKGTNTYGAVAGYVSGLFLRITGGEPYLYLNKLKSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSGSRLDKSKVINSALELLNGVGIEGLTTRKLAQKLGVEQPTLYWHVKNKRALLDALPIEMLDRHHTHSCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 7 - DNA, 2370 bp, artificial
  Cholinetransporter-SNAP25-tTA (3)(pCMV-Tet3G)
gaattcaccatggctttccatgtggaaggactgatagctatcatcgtgttctaccttctaattttgctggttggaatatgggctgcctggagaaccaaaaacagtggcagcgcagaagagcgcagcgaagccatcatagttggtggccgagatattggtttattggttggtggatttaccatgacagctacctgggtcggaggagggtatatcaatggcacagctgaagcagtttatgtaccaggttatggcctagcttgggctcaggcaccaattggatattctcttagtctgattttaggtggcctgttctttgcaaaacctatgcgttcaaaggggtatgtgaccatgttagacccgtttcagcaaatctatggaaaacgcatgggcggactcctgtttattcctgcactgatgggagaaatgttctgggctgcagcaattttctctgctttgggagccaccatcagcgtgatcatcgatgtggatatgcacatttctgtcatcatctctgcactcattgccactctgtacacactggtgggagggctctattctgtggcctacactgatgtcgttcagctcttttgcatttttgtagggctgtggatcagcgtcccctttgcattgtcacatcctgcagtcgcagacatcgggttcactgctgtgcatgccaaataccaaaagccgtggctgggaactgttgactcatctgaagtctactcttggcttgatagttttctgttgttgatgctgggtggaatcccatggcaagcatactttcagagggttctctcttcttcctcagccacctatgctcaagtgctgtccttcctggcagctttcgggtgcctggtgatggccatcccagccatactcattggggccattggagcatcaacagactggaaccagactgcatatgggcttccagatcccaagactacagaagaggcagacatgattttaccaattgttctgcagtatctctgccctgtgtatatttctttctttggtcttggtgcagtttctgctgctgttatgtcatcagcagattcttccatcttgtcagcaagttccatgtttgcacggaacatctaccagctttccttcagacaaaatgcttcggacaaagaaatcgtttgggttatgcgaatcacagtgtttgtgtttggagcatctgcaacagccatggccttgctgacgaaaactgtgtatgggctctggtacctcagttctgaccttgtttacatcgttatcttcccccagctgctttgtgtactctttgttaagggaaccaacacctatggggccgtggcaggttatgtttctggcctcttcctgagaataactggaggggagccatatctgtatcttcagcccttgatcttctaccctggctattaccctgatgataatggtatatataatcagaaatttccatttaaaacacttgccatggttacatcattcttaaccaacatttgcatctcctatctagccaagtatctatttgaaagtggaaccttgccacctaaattagatgtatttgatgctgttgttgcaagacacagtgaagaaaacatggataagacaattcttgtcaaaaatgaaaatattaaattagatgaacttgcacttgtgaagccacgacagagcatgaccctcagctcaactttcaccaataaagaggccttccttgatgttgattccagtccagaagggtctgggactgaagataatttacaggccgaagacgcagacatgcgcaatgagctggaggagatgcagcgaagggctgaccagttggctgatgagtcgctggaaagcacccgtcgtatgctgcaactggttgaagagagtaaagatgctggtatcaggactttggttatgttggatgaacaaggagaacaactggaacgcattgaggaagggatggaccaaatcaataaggacatgaaagaagcagaaaagaatttgacggacctaggaaaattctgcgggctttgtgtgtgtccctgtaacaagcttaaatcaagtgatgcttacaaaaaagcctggggcaataatcaggacggagtggtggccagccagcctgctcgtgtagtggacgaacgggagcagatggccatcagtggcggcttcatccgcagggtaacaaatgatgcccgagaaaatgaaatggatgaaaacctagagcaggtgagcggcatcatcgggaacctccgtcacatggccctggatatgggcaatgagatcgatacacagaatcgccagatcgacaggatcatggagaaggctgattccaacaaaaccagaattgatgaggccaaccaacgtgcaacaaagatgctgggaagtggttctaga

SEQ ID NO: 8 - protein, 1032 aa, artificial [sequence omitted]

SEQ ID NO: 9 - DNA, 603 bp, artificial
  Synaptotagmin-SNAP25-tTA(pCMV-Tet3G)
gaattcaccatgaggaacattttcaagaggaaccaggagcctattgtggctcctgccaccaccaccgccacgatgcccattggacccgtggacaactccactgagagtgggggtgctggggagagccaggaggacatgtttgccaaactgaaggagaagttattcaatgagataaacaagattcccttaccaccctgggcactgatcgccattgctgtggttgctgggctcctgcttctcacctgctgcttctgcatcaagcttaaatcaagtgatgcttacaaaaaagcctggggcaataatcaggacggagtggtggccagccagcctgctcgtgtagtggacgaacgggagcagatggccatcagtggcggcttcatccgcagggtaacaaatgatgcccgagaaaatgaaatggatgaaaacctagagcaggtgagcggcatcatcgggaacctccgtcacatggccctggatatgggcaatgagatcgatacacagaatcgccagatcgacaggatcatggagaaggctgattccaacaaaaccagaattgatgaggccaaccaacgtgcaacaaagatgctgggaagtggttctaga

SEQ ID NO: 10 - protein, 443 aa, artificial
  Synaptotagmin-SNAP25-tTA(pCMV-Tet3G)
MRNIFKRNQEPIVAPATTTATMPIGPVDNSTESGGAGESQEDMFAKLKEKLFNEINKIPLPPWALIAIAVVAGLLLLTCCFCIKLKSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSGSRLDKSKVINSALELLNGVGIEGLTTRKLAQKLGVEQPTLYWHVKNKRALLDALPIEMLDRHHTHSCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 11 - DNA, 3600 bp, artificial
  mAN02-SNAP25(pcDNA3.1)
cttaagatgcccgccctgcgccccgctctgctgtgggcgctgctggcgctctggctgtgctgcgcggcccccgcgcatgcattgcagtgtcgagatcctgccacctgtctgagccagtattgtgccgacaaagctcgggatggcgtctgtgatgaggcctgcaacagccatgcctgccagtgggatgggggtgactgttctctcaccatggagaacccctgggccaactgctcctccccacttccctgctgggattatatcaacaaccagtgtgatgagctgtgcaacacggtcgagtgcctgtttgacaactttgaatgccaggggaacagcaagacatgcaagtatgacaaatactgtgcagaccacttcaaagacaaccactgtgaccaggggtgcaacagtgaggagtgtggttgggatgggctggactgtgctgctgaccaacctgagaacctggcagaaggtaccctggttattgtggtattgatgccacctgaacaactgctccaggatgctcgcagcttcttgcgggcactgggtaccctgctccacaccaacctgcgcattaagcgggactcccagggggaactcatggtgtacccctattatggtgagaagtcagctgctatgaagaaacagaggatgacacgcagatcccttcctggtgaacaagaacaggaggtggctggctctaaagtctttctggaaattgacaaccgccagtgtgttcaagactcagaccactgcttcaagaacacggatgcagcagcagctctcctggcctctcacgccatacaggggaccctgtcataccctcttgtgtctgtcgtcagtgaatccctgactccagaacgcactcagctcctctatctccttgctgttgctgttgtcatcattctgtttattattctgctgggggtaatcaacaagcttaaatcaagtgatgcttacaaaaaagcctggggcaataatcaggacggagtggtggccagccagcctgctcgtgtagtggacgaacgggagcagatggccatcagtggcggcttcatccgcagggtaacaaatgatgcccgagaaaatgaaatggatgaaaacctagagcaggtgagcggcatcatcgggaacctccgtcacatggccctggatatgggcaatgagatcgatacacagaatcgccagatcgacaggatcatggagaaggctgattccaacaaaaccagaattgatgaggccaaccaacgtgcaacaaagatgctgggaagtggtatggcaaaacgaaagcgtaagcatggctctctctggctgcctgaaggtttcactcttcgccgagatgcaagcaatcacaagcgtcgtgagccagtgggacaggatgctgtggggctgaaaaatctctcagtgcaagtctcagaagctaacctaattggtactggaacaagtgaacactgggtcgatgatgaagggccccagccaaagaaagtaaaggctgaagatgaggccttactctcagaagaagatgaccccattgatcgacggccatggacacagcagcaccttgaagctgcagacatccgtaggacaccatcgctggctctcacccctcctcaggcagagcaggaggtggatgtgttagatgtgaatgtccgtggcccagatggctgcaccccattgatgttggcttctctccgaggaggcagctcagatttgagtgatgaagatgaagatgcagaggactcttctgctaacatcatcacagacttggtctaccagggtgccagcctccaggcccagacagaccggactggtgagatggccctgcaccttgcagcccgctactcacgggctgatgctgccaagcgtctcctggatgcaggtgcagatgccaatgcccaggacaacatgggccgctgtccactccatgctgcagtggcagctgatgcccaaggtgtcttccagattctgattcgcaaccgagtaactgatctagatgccaggatgaatgatggtactacacccctgatcctggctgcccgcctggctgtggagggaatggtggcagaactgatcaactgccaagcggatgtgaatgcagtggatgaccatggaaaatctgctcttcactgggcagctgctgtcaataatgtggaggcaactcttttgttgttgaaaaatggggccaaccgagacatgcaggacaacaaggaagagacacctctgtttcttgctgcccgggaggggagctatgaagcagccaagatcctgttagaccattttgccaatcgagacatcacagaccatatggatcgtcttccccgggatgtggctcgggatcgcatgcaccatgacattgtgcgccttctggatgaatacaatgtgaccccaagccctccaggcaccgtgttgacttctgctctctcacctgtcatctgtgggcccaacagatctttcctcagcctgaagcacaccccaatgggcaagaagtctagacggcccagtgccaagagtaccatgcctactagcctccctaaccttgccaaggaggcaaaggatgccaagggtagtaggaggaagaagtctctgagtgagaaggtccaactgtctgagagttcagtaactttatcccctgttgattccctagaatctcctcacacgtatgtttccgacaccacatcctctccaatgattacatcccctgggatcttacaggcctcacccaaccctatgttggccactgccgcccctcctgccccagtccatgcccagcatgcactatctttttctaaccttcatgaaatgcagcctttggcacatggggccagcactgtgcttccctcagtgagccagttgctatcccaccaccacattgtgtctccaggcagtggcagtgctggaagcttgagtaggctccatccagtcccagtcccagcagattggatgaaccgcatggaggtgaatgagacccagtacaatgagatgtttggtatggtcctggctccagctgagggcacccatcctggcatagctccccagagcaggccacctgaagggaagcacataaccacccctcgggagcccttgccccccattgtgactttccagctcatccctaaaggcagtattgcccaaccagcgggggctccccagcctcagtccacctgccctccagctgttgcgggccccctgcccaccatgtaccagattccagaaatggcccgtttgcccagtgtggctttccccactgccatgatgccccagcaggacgggcaggtagctcagaccattctcccagcctatcatcctttcccagcctctgtgggcaagtaccccacacccccttcacagcacagttatgcttcctcaaatgctgctgagcgaacacccagtcacagtggtcacctccagggtgagcatccctacctgacaccatccccagagtctcctgaccagtggtcaagttcatcaccccactctgcttctgactggtcagatgtgaccaccagccctacccctgggggtgctggaggaggtcagcggggacctgggacacacatgtctgagccaccacacaacaacatgcaggtttatgcgtgataatctaga

SEQ ID NO: 12 - protein, 1194 aa, artificial
  mAN02-SNAP25(pcDNA3.1)
MPALRPALLWALLALWLCCAAPAHALQCRDPATCLSQYCADKARDGVCDEACNSHACQWDGGDCSLTMENPWANCSSPLPCWDYINNQCDELCNTVECLFDNFECQGNSKTCKYDKYCADHFKDNHCDQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVINKLKSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSGMAKRKRKHGSLWLPEGFTLRRDASNHKRREPVGQDAVGLKNLSVQVSEANLIGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 13 - DNA, 633 bp, artificial
  SNAP25-tTA(pCMV-Tet3G)
gaattcaccatggccgaagacgcagacatgcgcaatgagctggaggagatgcagcgaagggctgaccagttggctgatgagtcgctggaaagcacccgtcgtatgctgcaactggttgaagagagtaaagatgctggtatcaggactttggttatgttggatgaacaaggagaacaactggaacgcattgaggaagggatggaccaaatcaataaggacatgaaagaagcagaaaagaatttgacggacctaggaaaattctgcgggctttgtgtgtgtccctgtaacaagcttaaatcaagtgatgcttacaaaaaagcctggggcaataatcaggacggagtggtggccagccagcctgctcgtgtagtggacgaacgggagcagatggccatcagtggcggcttcatccgcagggtaacaaatgatgcccgagaaaatgaaatggatgaaaacctagagcaggtgagcggcatcatcgggaacctccgtcacatggccctggatatgggcaatgagatcgatacacagaatcgccagatcgacaggatcatggagaaggctgattccaacaaaaccagaattgatgaggccaaccaacgtgcaacaaagatgctgggaagtggttctaga

SEQ ID NO: 14 - protein, 453 aa, artificial
  SNAP25-tTA(pCMV-Tet3G)
MAEDADMRNELEEMQRRADQLADESLESTRRMLQLVEESKDAGIRTLVMLDEQGEQLERIEEGMDQINKDMKEAEKNLTDLGKFCGLCVCPCNKLKSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSGSRLDKSKVINSALELLNGVGIEGLTTRKLAQKLGVEQPTLYWHVKNKRALLDALPIEMLDRHHTHSCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 15 - DNA, 1290 bp, artificial
  GST-SNAP25-tTA (2)(pCMV-Tet3G)
gaattcaccatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatgccgaagacgcagacatgcgcaatgagctggaggagatgcagcgaagggctgaccagttggctgatgagtcgctggaaagcacccgtcgtatgctgcaactggttgaagagagtaaagatgctggtatcaggactttggttatgttggatgaacaaggagaacaactggaacgcattgaggaagggatggaccaaatcaataaggacatgaaagaagcagaaaagaatttgacggacctaggaaaattctgcgggctttgtgtgtgtccctgtaacaagcttaaatcaagtgatgcttacaaaaaagcctggggcaataatcaggacggagtggtggccagccagcctgctcgtgtagtggacgaacgggagcagatggccatcagtggcggcttcatccgcagggtaacaaatgatgcccgagaaaatgaaatggatgaaaacctagagcaggtgagcggcatcatcgggaacctccgtcacatggccctggatatgggcaatgagatcgatacacagaatcgccagatcgacaggatcatggagaaggctgattccaacaaaaccagaattgatgaggccaaccaacgtgcaacaaagatgctgggaagtggttctaga

SEQ ID NO: 16 - protein, 672 aa, artificial
  GST-SNAP25-tTA (2)(pCMV-Tet3G)
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDAEDADMRNELEEMQRRADQLADESLESTRRMLQLVEESKDAGIRTLVMLDEQGEQLERIEEGMDQINKDMKEAEKNLTDLGKFCGLCVCPCNKLKSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSGSRLDKSKVINSALELLNGVGIEGLTTRKLAQKLGVEQPTLYWHVKNKRALLDALPIEMLDRHHTHSCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 17 - DNA, 2094 bp, artificial
  GFP-SNAP25-tTA(pcDNA3.1)
cttaaggccaccatggccagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactggagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaagccgaagacgcagacatgcgcaatgagctggaggagatgcagcgaagggctgaccagttggctgatgagtcgctggaaagcacccgtcgtatgctgcaactggttgaagagagtaaagatgctggtatcaggactttggttatgttggatgaacaaggagaacaactggaacgcattgaggaagggatggaccaaatcaataaggacatgaaagaagcagaaaagaatttgacggacctaggaaaattctgcgggctttgtgtgtgtccctgtaacaagcttaaatcaagtgatgcttacaaaaaagcctggggcaataatcaggacggagtggtggccagccagcctgctcgtgtagtggacgaacgggagcagatggccatcagtggcggcttcatccgcagggtaacaaatgatgcccgagaaaatgaaatggatgaaaacctagagcaggtgagcggcatcatcgggaacctccgtcacatggccctggatatgggcaatgagatcgatacacagaatcgccagatcgacaggatcatggagaaggctgattccaacaaaaccagaattgatgaggccaaccaacgtgcaacaaagatgctgggaagtggttctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaactcgag

SEQ ID NO: 18 - protein, 691 aa, artificial
  GFP-SNAP25-tTA(pcDNA3.1)
MASKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYKAEDADMRNELEEMQRRADQLADESLESTRRMLQLVEESKDAGIRTLVMLDEQGEQLERIEEGMDQINKDMKEAEKNLTDLGKFCGLCVCPCNKLKSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSGSRLDKSKVINSALELLNGVGIEGLTTRKLAQKLGVEQPTLYWHVKNKRALLDALPIEMLDRHHTHSCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 19 - DNA, 1731 bp, artificial
  MBP-SNAP25-tTA (2)(pCMV-Tet3G)
gaattcaccatgaagaccgaggagggcaagctggtgatctggatcaacggcgacaagggctacaacggcctggccgaggtgggcaagaagttcgagaaggacaccggcatcaaggtgaccgtggagcaccccgacaagctggaggagaagttcccccaggtggccgccaccggcgacggccccgacatcatcttctgggcccacgacagattcggcggctacgcccagagcggcctgctggccgagatcacccccgacaaggccttccaggacaagctgtaccccttcacctgggacgccgtgagatacaacggcaagctgatcgcctaccccatcgccgtggaggccctgagcctgatctacaacaaggacctgctgcccaacccccccaagacctgggaggagatccccgccctggacaaggagctgaaggccaagggcaagagcgccctgatgttcaacctgcaggagccctacttcacctggcccctgatcgccgccgacggcggctacgccttcaagtacgagaacggcaagtacgacatcaaggacgtgggcgtggacaacgccggcgccaaggccggcctgaccttcctggtggacctgatcaagaacaagcacatgaacgccgacaccgactacagcatcgccgaggccgccttcaacaagggcgagaccgccatgaccatcaacggcccctgggcctggagcaacatcgacaccagcaaggtgaactacggcgtgaccgtgctgcccaccttcaagggccagcccagcaagcccttcgtgggcgtgctgagcgccggcatcaacgccgccagccccaacaaggagctggccaaggagttcctggagaactacctgctgaccgacgagggcctggaggccgtgaacaaggacaagcccctgggcgccgtggccctgaagagctacgaggaggagctggccaaggaccccagaatcgccgccaccatggagaacgcccagaagggcgagatcatgcccaacatcccccagatgagcgccttctggtacgccgtgagaaccgccgtgatcaacgccgccagcggcagacagaccgtggacgaggccctgaaggacgcccagaccgccgaagacgcagacatgcgcaatgagctggaggagatgcagcgaagggctgaccagttggctgatgagtcgctggaaagcacccgtcgtatgctgcaactggttgaagagagtaaagatgctggtatcaggactttggttatgttggatgaacaaggagaacaactggaacgcattgaggaagggatggaccaaatcaataaggacatgaaagaagcagaaaagaatttgacggacctaggaaaattctgcgggctttgtgtgtgtccctgtaacaagcttaaatcaagtgatgcttacaaaaaagcctggggcaataatcaggacggagtggtggccagccagcctgctcgtgtagtggacgaacgggagcagatggccatcagtggcggcttcatccgcagggtaacaaatgatgcccgagaaaatgaaatggatgaaaacctagagcaggtgagcggcatcatcgggaacctccgtcacatggccctggatatgggcaatgagatcgatacacagaatcgccagatcgacaggatcatggagaaggctgattccaacaaaaccagaattgatgaggccaaccaacgtgcaacaaagatgctgggaagtggttctaga

SEQ ID NO: 20 - protein, 819 aa, artificial
  MBP-SNAP25-tTA (2) (pCMV-Tet3G)
MKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTAEDADMRNELEEMQRRADQLADESLESTRRMLQLVEESKDAGIRTLVMLDEQGEQLERIEEGMDQINKDMKEAEKNLTDLGKFCGLCVCPCNKLKSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSGSRLDKSKVINSALELLNGVGIEGLTTRKLAQKLGVEQPTLYWHVKNKRALLDALPIEMLDRHHTHSCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 21 - DNA, 1089 bp, artificial
  CLAT-SNAP25-tTA (1)(pCMV-Tet3G)
gaattcaccatggctgagctggatccgttcggcgcccctgccggcgcccctggcggtcccgcgctggggaacggagtggccggcgccggcgaagaagacccggctgcggccttcttggcgcagcaagagagcgagattgcgggcatcgagaacgacgaggccttcgccatcctggacggcggcgcccccgggccccagccgcacggcgagccgccggggggtccggatgctgttgatggagtaatgaatggtgaatactaccaggaaagtaatggtccaacagacagttatgcagctatttcacaagtggatcgattgcagtcagagcctgaaagtatccgtaaatggagagaagaacaaatggaacgcttggaagcccttgatgccaattctcggaagcaagaagcagagtggaaagaaaaggcaataaaggagctagaagaatggtatgcaagacaggacgagcagctacagaaaacaaaagcaaacaacagggtggcagatgaagctttctacaaacaacccttcgctgacgtgattggttatgtcacaaacataaaccatccttgctacagcctagaacaggcagcagaagaagcctttgtaaatgacattgacgagtcgtccccaggcactgagtgggaacgggtggcccggctgtgtgactttaaccccaagtctagcaagcaggccaaagatgtctcccgcatgcgctcagtcctcatctccctcaagcaggccccgctggtgcactcaagtgatgcttacaaaaaagcctggggcaataatcaggacggagtggtggccagccagcctgctcgtgtagtggacgaacgggagcagatggccatcagtggcggcttcatccgcagggtaacaaatgatgcccgagaaaatgaaatggatgaaaacctagagcaggtgagcggcatcatcgggaacctccgtcacatggccctggatatgggcaatgagatcgatacacagaatcgccagatcgacaggatcatggagaaggctgattccaacaaaaccagaattgatgaggccaaccaacgtgcaacaaagatgctgggaagtggttctaga

SEQ ID NO: 22 - protein, 605 aa, artificial
  CLAT-SNAP25-tTA (1)(pCMV-Tet3G)
MAELDPFGAPAGAPGGPALGNGVAGAGEEDPAAAFLAQQESEIAGIENDEAFAILDGGAPGPQPHGEPPGGPDAVDGVMNGEYYQESNGPTDSYAAISQVDRLQSEPESIRKWREEQMERLEALDANSRKQEAEWKEKAIKELEEWYARQDEQLQKTKANNRVADEAFYKQPFADVIGYVTNINHPCYSLEQAAEEAFVNDIDESSPGTEWERVARLCDFNPKSSKQAKDVSRMRSVLISLKQAPLVHSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSGSRLDKSKVINSALELLNGVGIEGLTTRKLAQKLGVEQPTLYWHVKNKRALLDALPIEMLDRHHTHSCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 23 - DNA, 3731 bp, artificial
  tTA-SNAP25-SV2 (pCMV-Tet3G)
gaattcaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccggggccggcgccggcagcggagtggtggccagccagcctgctcgtgtagtggacgaacgggagcagatggccatcagtggcggcttcatccgcagggtaacaaatgatgcccgagaaaatgaaatggatgaaaacctagagcaggtgagcggcatcatcgggaacctccgtcacatggccctggatatgggcaatgagatcgatacacagaatcgccagatcgacaggatcatggagaaggctgattccaacaaaaccagaattgatgaggccaaccaacgtgcaacaaagatgctgggaagtggtgccggcgccggcagcgaagactcttacaaggataggacttcactgatgaagggtgccaaggacattgccagagaggtgaagaaacaaacagtaaagaaggtgaatcaagctgtggaccgagcccaggatgaatacacccagaggtcctacagtcggttccaagatgaagaagatgatgatgactactacccggctggagaaacctataatggtgaggccaacgatgacgaaggctcaagtgaagccactgaggggcatgatgaagatgatgagatctatgagggggagtatcagggcatccccagtatgaaccaagcgaaggacagcatcgtgtcagtggggcagcccaagggcgatgagtacaaggaccgacgggagctggaatcagaaaggagagctgacgaggaagagttagcccagcagtatgagctgataatccaagaatgcggtcatggtcgttttcagtgggcccttttcttcgtcctgggcatggctcttatggcagacggtgtagaggtgtttgtcgttggcttcgtgttacccagtgctgagacagacctctgcatcccaaattcaggatctggatggctaggcagcatagtgtacctcgggatgatggtgggggcgttcttctggggaggactggcagacaaagtgggaaggaaacagtctcttctgatttgcatgtctgtcaacggattctttgccttcctttcttcatttgtccaaggttatggcttctttctcttctgtcgcttactttctggattcgggattggaggagccatacccactgtgttctcgtactttgctgaagtcctggcccgggaaaagcggggcgaacacttgagctggctctgcatgttctggatgatcggtggcatctacgcctctgccatggcctgggccatcatcccgcactacgggtggagcttcagcatgggatcggcctaccagtttcacagttggcgtgtgtttgtcatcgtctgtgcactcccctgtgtctcctccgtggtggccctcacattcatgcctgaaagcccacgattcttgttggaggttggaaaacatgatgaagcttggatgattctgaagttaattcatgacaccaacatgagagcccggggtcagcctgagaaggtcttcacggtaaacaaaataaaaactcctaaacaaatagatgagctgattgaaattgagagtgacacaggaacatggtataggaggtgttttgttcggatccgcaccgagctgtacggaatttggttgacttttatgagatgtttcaactacccagtcagggataatacaataaagcttacaattgtttggttcaccctgtcctttgggtactatggattatccgtttggttccctgatgtcattaaacctctgcagtccgatgaatatgcattgctaaccagaaatgtggagagagataaatatgcaaatttcactattaactttacaatggaaaatcagattcatactggaatggaatacgacaatggcagattcataggggtcaagttcaaatctgtaactttcaaagactctgtttttaagtcctgcacctttgaggatgtaacttcagtgaacacctacttcaagaactgcacatttattgacactgtttttgacaacacagattttgagccatataaattcattgacagtgaatttaaaaactgctcgttttttcacaacaagacgggatgtcagattacctttgatgatgactatagtgcctactggatttattttgtcaactttctggggacattggcagtattgccagggaacattgtgtctgctctgctgatggacagaattgggcgcttaacaatgctaggtggctctatggtgctttcggggatcagctgtttcttcctttggttcggcaccagtgaatccatgatgataggcatgctgtgtctgtacaatggattgaccatctcagcctggaactctcttgacgtggtcactgtggaactgtaccccacagaccggagggcaacaggctttggcttcttaaatgcgctatgcaaggcagcagccgtcctgggaaacttaatatttggctctctggtcagcatcaccaaatcaatccccatcctgctggcttctactgtgctcgtgtgtggaggactcgttgggctgtgcctgcctgacacacgaacccaggttctgtaactaagtaaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtcctggccggaccacgctatctgtgcaaggtccccggccccggacgcgcgctccatgagcagagcgcccgccgccgaggcgaagactcgggcggcgccctgcccgtcccaccaggtcaacaggcggtaaccggcctcttcatcgggaatgcgcgcgaccttcagcatcgccggcatgtccccctggcggacgggaagtatccagctcgaccaagctt

SEQ ID NO: 24 - protein, 1079 aa, artificial [sequence omitted]

SEQ ID NO: 25 - DNA, 3311 bp, artificial
  tTA-SNAP25-Cholinetransporter(pCMV-Tet3G)
gaattcaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccggggccggcgccggcagcggagtggtggccagccagcctgctcgtgtagtggacgaacgggagcagatggccatcagtggcggcttcatccgcagggtaacaaatgatgcccgagaaaatgaaatggatgaaaacctagagcaggtgagcggcatcatcgggaacctccgtcacatggccctggatatgggcaatgagatcgatacacagaatcgccagatcgacaggatcatggagaaggctgattccaacaaaaccagaattgatgaggccaaccaacgtgcaacaaagatgctgggaagtggtgccggcgccggcagcggcgccggcgccggcagcgctttccatgtggaaggactgatagctatcatcgtgttctaccttctaattttgctggttggaatatgggctgcctggagaaccaaaaacagtggcagcgcagaagagcgcagcgaagccatcatagttggtggccgagatattggtttattggttggtggatttaccatgacagctacctgggtcggaggagggtatatcaatggcacagctgaagcagtttatgtaccaggttatggcctagcttgggctcaggcaccaattggatattctcttagtctgattttaggtggcctgttctttgcaaaacctatgcgttcaaaggggtatgtgaccatgttagacccgtttcagcaaatctatggaaaacgcatgggcggactcctgtttattcctgcactgatgggagaaatgttctgggctgcagcaattttctctgctttgggagccaccatcagcgtgatcatcgatgtggatatgcacatttctgtcatcatctctgcactcattgccactctgtacacactggtgggagggctctattctgtggcctacactgatgtcgttcagctcttttgcatttttgtagggctgtggatcagcgtcccctttgcattgtcacatcctgcagtcgcagacatcgggttcactgctgtgcatgccaaataccaaaagccgtggctgggaactgttgactcatctgaagtctactcttggcttgatagttttctgttgttgatgctgggtggaatcccatggcaagcatactttcagagggttctctcttcttcctcagccacctatgctcaagtgctgtccttcctggcagctttcgggtgcctggtgatggccatcccagccatactcattggggccattggagcatcaacagactggaaccagactgcatatgggcttccagatcccaagactacagaagaggcagacatgattttaccaattgttctgcagtatctctgccctgtgtatatttctttctttggtcttggtgcagtttctgctgctgttatgtcatcagcagattcttccatcttgtcagcaagttccatgtttgcacggaacatctaccagctttccttcagacaaaatgcttcggacaaagaaatcgtttgggttatgcgaatcacagtgtttgtgtttggagcatctgcaacagccatggccttgctgacgaaaactgtgtatgggctctggtacctcagttctgaccttgtttacatcgttatcttcccccagctgctttgtgtactctttgttaagggaaccaacacctatggggccgtggcaggttatgtttctggcctcttcctgagaataactggaggggagccatatctgtatcttcagcccttgatcttctaccctggctattaccctgatgataatggtatatataatcagaaatttccatttaaaacacttgccatggttacatcattcttaaccaacatttgcatctcctatctagccaagtatctatttgaaagtggaaccttgccacctaaattagatgtatttgatgctgttgttgcaagacacagtgaagaaaacatggataagacaattcttgtcaaaaatgaaaatattaaattagatgaacttgcacttgtgaagccacgacagagcatgaccctcagctcaactttcaccaataaagaggccttccttgatgttgattccagtccagaagggtctgggactgaagataatttacagtaactaagtaaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtcctggccggaccacgctatctgtgcaaggtccccggccccggacgcgcgctccatgagcagagcgcccgccgccgaggcgaagactcgggcggcgccctgcccgtcccaccaggtcaacaggcggtaaccggcctcttcatcgggaatgcgcgcgaccttcagcatcgccggcatgtccccctggcggacgggaagtatccagctcgaccaagctt

SEQ ID NO: 26 - protein, 939 aa, artificial
  tTA-SNAP25-Cholinetransporter(pCMV-Tet3G)
MSRLDKSKVINSALELLNGVGIEGLTTRKLAQKLGVEQPTLYWHVKNKRALLDALPIEMLDRHHTHSCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPGAGAGSGVVASQPARVVDEREQMAISGGFIRRVTNDARENEMDENLEQVSGIIGNLRHMALDMGNEIDTQNRQIDRIMEKADSNKTRIDEANQRATKMLGSGAGAGSGAGAGSAFHVEGLIAIIVFYLLILLVGIWAAWRTKNSGSAEERSEAIIVGGRDIGLLVGGFTMTATWVGGGYINGTAEAVYVPGYGLAWAQAPIGYSLSLILGGLFFAKPMRSKGYVTMLDPFQQIYGKRMGGLLFIPALMGEMFWAAAIFSALGATISVIIDVDMHISVIISALIATLYTLVGGLYSVAYTDVVQLFCIFVGLWISVPFALSHPAVADIGFTAVHAKYQKPWLGTVDSSEVYSWLDSFLLLMLGGIPWQAYFQRVLSSSSATYAQVLSFLAAFGCLVMAIPAILIGAIGASTDWNQTAYGLPDPKTTEEADMILPIVLQYLCPVYISFFGLGAVSAAVMSSADSSILSASSMFARNIYQLSFRQNASDKEIVWVMRITVFVFGASATAMALLTKTVYGLWYLSSDLVYIVIFPQLLCVLFVKGTNTYGAVAGYVSGLFLRITGGEPYLYLQPLIFYPGYYPDDNGIYNQKFPFKTLAMVTSFLTNICISYLAKYLFESGTLPPKLDVFDAVVARHSEENMDKTILVKNENIKLDELALVKPRQSMTLSSTFTNKEAFLDVDSSPEGSGTEDNLQ

SEQ ID NO: 27 - DNA, 2198 bp, artificial
  tTA-SNAP25-GST(pCMV-Tet3G)
gaattcaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccggggccggcgccggcagcggagtggtggccagccagcctgctcgtgtagtggacgaacgggagcagatggccatcagtggcggcttcatccgcagggtaacaaatgatgcccgagaaaatgaaatggatgaaaacctagagcaggtgagcggcatcatcgggaacctccgtcacatggccctggatatgggcaatgagatcgatacacagaatcgccagatcgacaggatcatggagaaggctgattccaacaaaaccagaattgatgaggccaaccaacgtgcaacaaagatgctgggaagtggttcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggattaactaagtaaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtcctggccggaccacgctatctgtgcaaggtccccggccccggacgcgcgctccatgagcagagcgcccgccgccgaggcgaagactcgggcggcgccctgcccgtcccaccaggtcaacaggcggtaaccggcctcttcatcgggaatgcgcgcgaccttcagcatcgccggcatgtccccctggcggacgggaagtatccagctcgaccaagctt

SEQ ID NO: 28 - protein, 569 aa, artificial
  tTA-SNAP25-GST(pCMV-Tet3G)
MSRLDKSKVINSALELLNGVGIEGLTTRKLAQKLGVEQPTLYWHVKNKRALLDALPIEMLDRHHTHSCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPGAGAGSGVVASQPARVVDEREQMAISGGFIRRVTNDARENEMDENLEQVSGIIGNLRHMALDMGNEIDTQNRQIDRIMEKADSNKTRIDEANQRATKMLGSGMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSD

SEQ ID NO: 29 - DNA, 2255 bp, artificial
  tTA-SNAP25-GFP(pCMV-Tet3G)
gaattcaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccggggccggcgccggcagcggagtggtggccagccagcctgctcgtgtagtggacgaacgggagcagatggccatcagtggcggcttcatccgcagggtaacaaatgatgcccgagaaaatgaaatggatgaaaacctagagcaggtgagcggcatcatcgggaacctccgtcacatggccctggatatgggcaatgagatcgatacacagaatcgccagatcgacaggatcatggagaaggctgattccaacaaaaccagaattgatgaggccaaccaacgtgcaacaaagatgctgggaagtggtgccagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataactaagtaaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtcctggccggaccacgctatctgtgcaaggtccccggccccggacgcgcgctccatgagcagagcgcccgccgccgaggcgaagactcgggcggcgccctgcccgtcccaccaggtcaacaggcggtaaccggcctcttcatcgggaatgcgcgcgaccttcagcatcgccggcatgtccccctggcggacgggaagtatccagctcgaccaagctt

SEQ ID NO: 30 - protein, 696 aa, artificial
  tTA-SNAP25-GFP(pCMV-Tet3G)
MSRLDKSKVINSALELLNGVGIEGLTTRKLAQKLGVEQPTLYWHVKNKRALLDALPIEMLDRHHTHSCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPGAGAGSGVVASQPARVVDEREQMAISGGFIRRVTNDARENEMDENLEQVSGIIGNLRHMALDMGNEIDTQNRQIDRIMEKADSNKTRIDEANQRATKMLGSGASKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYKAEDADMRNELEEMQRRADQLADESLESTRRMLQLVEESKDAGIRTLVMLDEQGEQLERIEEGMDQINKDMKEAEKNLTDLGKFCGLCVCPCNKLKSSDAYKKAWGNNQD

SEQ ID NO: 31 - protein, 444 aa, artificial
  SNAP110-pTET-tTAK
SSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSGSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 32 - protein, 10 aa, artificial
  leucine-rich nuclear export signal (NES)
LXXXLXXLXL

SEQ ID NO: 33 - protein, 247 aa, artificial
  tetracycline dependent transactivator
SRLDKSKVINSALELLNGVGIEGLTTRKLAQKLGVEQPTLYWHVKNKRALLDALPIEMLDRHHTHSCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 34 - protein, 334 aa, artificial
  tetracycline dependent transactivator
SRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 35 - DNA, 22 bp, artificial
  forward primer Tet-element
agctgggagttgagcagcctac

SEQ ID NO: 36 - DNA, 22 bp, artificial
  reverse primer Tet-element
ggtgccgagatgcactttagcc

SEQ ID NO: 37 - DNA, 20 bp, artificial
  forward primer Luciferase
gaagagatacgccctggttc

SEQ ID NO: 38 - DNA, 20 bp, artificial
  reverse primer Luciferase
cgggaggtagatgagatgtg

(19) United States
(12) Patent Application Publication
(10) Pub. No.: US 2016/0289731 A1
(43) Pub. Date: Oct. 6, 2016

(54) MEANS AND METHODS FOR DETERMINATION OF BOTULINUM NEUROTOXIN BIOLOGICAL ACTIVITY

(71) Applicant: MERZ PHARMA GMBH & CO, KGAA, Frankfurt am Main (DE)

(72) Inventors: Karl-Heinz EISELE, FRANKFURT AM MAIN (DE); Klaus FINK, Koln (DE)

(21) Appl. No.: 14/442,180

(22) PCT Filed: Nov. 20, 2013
(86) PCT No.: PCT/EP2013/074276

§ 371 Date: May 12, 2015

Related U.S. Application Data
(60) Provisional application No. 61/729,048, filed on Nov. 21, 2012.

(30) Foreign Application Priority Data
Nov. 21, 2012 (EP) 12193560.5

(51) Int. Cl. C12Q 1/37; C07K 14/705; C12Q 1/66; C07K 14/47

(52) U.S. Cl. CPC C12Q 1/37; C07K 14/4702; C07K 14/705; C12Q 1/66; C07K 2319/50; C07K 2319/03; C07K 2319/80; G01N 2333/952

(57) ABSTRACT

The present invention is concerned with means and methods for determining neurotoxin activity. Specifically, it relates to a polynucleotide encoding a fusion polypeptide comprising (i) a transcription factor domain and (ii) a cytoplasmic retention domain, separated by a linker comprising a neurotoxin cleavage site and to a fusion polypeptide encoded by the polynucleotide of the invention. Also contemplated is a vector comprising the polynucleotide of the invention and a host cell comprising the polynucleotide, vector or fusion polypeptide of the invention. Moreover, envisaged is a method for determining neurotoxin activity in a sample. In addition, the invention pertains to the use of the polynucleotide, vector, fusion polypeptide or host cell of the invention for determining neurotoxin activity in a sample. Finally, the invention relates to a kit for determining neurotoxin activity comprising the polynucleotide, vector, fusion polypeptide or host cell of the invention.

The present invention is concerned with means and methods for determining neurotoxin activity. Specifically, it relates to a polynucleotide encoding a fusion polypeptide comprising (i) a transcription factor domain and (ii) a cytoplasmic retention domain, separated by a linker comprising a neurotoxin cleavage site as well as to a fusion polypeptide encoded by the polynucleotide of the invention. Also contemplated is a vector comprising the polynucleotide of the invention and a host cell comprising the polynucleotide, vector or fusion polypeptide of the invention. Moreover, envisaged is a method for determining neurotoxin activity in a sample. In addition, the invention pertains to the use of the polynucleotide, vector, fusion polypeptide or host cell of the invention for determining neurotoxin activity in a sample. Finally, the invention relates to a kit for determining neurotoxin activity comprising the polynucleotide, vector, fusion polypeptide or host cell of the invention.

Clostridium botulinum and Clostridium tetani produce highly potent neurotoxins, i.e. botulinum toxins (BoNTs) and tetanus toxin (TeNT), respectively. These Clostridial neurotoxins specifically bind to neuronal cells and disrupt neurotransmitter release. Each toxin is synthesized as an inactive unprocessed approximately 150 kDa single-chain protein. The posttranslational processing involves formation of disulfide bridges, and limited proteolysis (nicking) by bacterial protease(s). Active dichain neurotoxin consists of two chains, an N-terminal light chain of approx. 50 kDa and a heavy chain of approx. 100 kDa linked by a disulfide bond. Neurotoxins structurally consist of three domains, i.e. the catalytic light chain, the heavy chain encompassing the translocation domain (N-terminal half) and the receptor binding domain (C-terminal half), see Krieglstein 1990, Eur. J. Biochem. 188, 39; Krieglstein 1991, Eur. J. Biochem. 202, 41; Krieglstein 1994, J. Protein Chem. 13, 49.

Clostridium botulinum secretes seven antigenically distinct serotypes designated A to G of the BoNTs. All serotypes together with the related TeNT secreted by Clostridium tetani, are zinc (Zn²⁺)-dependent endoproteases that block synaptic exocytosis by cleaving SNARE proteins and, in particular, SNAP-25. BoNTs cause, inter alia, the flaccid muscular paralysis seen in botulism and tetanus, see Fischer 2007, PNAS 104, 10447.

Despite its toxic effects, BoNTs have been used as therapeutic agents in a large number of diseases. BoNT serotype A (BoNT/A) was approved for human use in the United States in 1989 for the treatment of strabism, blepharospasm, and other disorders. It is commercially available as a protein preparation, for example, under the tradename BOTOX (Allergan Inc) under the tradename DYSPORT (Ipsen Ltd). For therapeutic application the complex is injected directly into the muscle to be treated. At physiological pH, the toxin is released from the protein complex and the desired pharmacological effect takes place. An improved BoNT/A preparation being free of complexing proteins is available under the tradename XEOMIN (Merz Pharmaceuticals GmbH).

BoNTs, in principle, weaken voluntary muscle strength and are, therefore, effective therapeutic agents for the therapy of diseases such as strabism, focal dystonia, including cervical dystonia, and benign essential blepharospasm. They have been further shown to relief hemifacial spasm, and focal spasticity, and moreover, to be effective in a wide range of other indications, such as gastrointestinal disorders, hyperhidrosis, and cosmetic wrinkle correction, see Jost 2007, Drugs 67, 669.

The determination of the biological activity is important as a safety measure, for quality control and for quantification purposes. WO 95/33850 describes methods for quantifying the biological activity of neurotoxins based on non-competitive assays. In these methods, a substrate comprising a cleavage site for the neurotoxin is coupled to a solid phase. The addition of the neurotoxin results in the cleavage of the substrate. The cleavage product is then detected by, e.g., a specific antibody conjugated to an enzyme. One drawback of such non-competitive assays is that the test results obtained may be inhomogeneous and therefore unreliable.

Further assays which have been developed for the determination of the biological activity of Botulinum neurotoxins pertain to, e.g., the determination of the time of survival of animals after intoxication, for example “time to death” (Lamanna C, Spero L, Schantz E J: Dependence of time to death on molecular size of botulinum toxin. Infect. Immun. 1970, 1:423-4; Kondo H, Shimizu T, Kubonoya M, Izumi N, Takahashi M, Sakaguchi G: Titration of botulinum toxins for lethal toxicity by intravenous injection into mice. Jpn. J. Med. Sci. Biol. 1984, 37:131-5; Boroff D A, Fleck U: Statistical analysis of a rapid in vivo method for the titration of the toxin of Clostridium botulinum. J. Bacteriol. 1966, 92:1580-1), the inhibition of organ functions of the living organism, such as paralysis of perspiratory glands (Ellies M: Experimental and clinical investigations on the inhibition of secretion of the major salivary glands with botulinum toxin A. Laryngorhinootologie 2003, 82:713-4; Monnier G, Tatu L, Parratte B, Cosson A, Michel F, Metton G: Sialorrhea, hyperhidrosis and botulinum toxin. Ann. Readapt. Med. Phys. 2003, 46:338-45), the determination of paralysis of isolated organs, such as HDA (Goschel H, Wohlfarth K, Frevert J, Dengler R, Bigalke H: Botulinum A toxin therapy: neutralizing and nonneutralizing antibodies—therapeutic consequences. Exp. Neurol. 1997, 147:96-102; HUGHES R, WHALER B C. Influence of nerve-ending activity and of drugs on the rate of paralysis of rat diaphragm preparations by Cl. botulinum type A toxin. J. Physiol. 1962, 160:221-33), cell based assays, such as SNAP-25 cleavage Western Blot assays using primary neurons (Pellett S, Tepp W H, Toth S I, Johnson E A: Comparison of the primary rat spinal cord cell (RSC) assay and the mouse bioassay for botulinum neurotoxin type A potency determination. J. Pharmacol. Toxicol. Methods 2010, 61:304-10) and in vitro tests, which, however, determine only single aspects of the biological activity of neurotoxins, such as binding assays or SNAP-25 cleavage assay (Evans E R, Skipper P J, Shone C C: An assay for botulinum toxin types A, B and F that requires both functional binding and catalytic activities within the neurotoxin. J. Appl. Microbiol. 2009, 107:1384-91; Habermann E: 125I-labeled neurotoxin from Clostridium botulinum A: preparation, binding to synaptosomes and ascent to the spinal cord. Naunyn Schmiedebergs Arch. Pharmacol. 1974, 281:47-56; Ekong T A, Feavers I M, Sesardic D: Recombinant SNAP-25 is an effective substrate for Clostridium botulinum type A toxin endopeptidase activity in vitro. Microbiology 1997, 143:3337-47).

The mouse LD50 assay is thus far the only reliable assay for quantifying the biological activity of neurotoxins and for assessing their therapeutic potential and/or their toxicity. Said assay is also accepted for quality control purposes during manufacture of neurotoxin. In the mouse LD50 bioassay, lethal and sub-lethal concentrations of a sample containing the neurotoxin polypeptide have to be injected into at least 120 animals. The number of killed animals over an observation period of 72 hours allows determining the neurotoxin polypeptide concentration in the sample. Apparent drawbacks of this assay are the high number of animals which will be sacrificed and the high level of stress and pain for said animals during the test.

In vitro assays which have been proposed so far are based on determining SNAP-25 cleavage in a cell free system or on neurotoxin exposure to primary neurons. However, these assays are less reliable and/or do not take into account all of the desired neurotoxin functions. Thus, at present, the LD50 bioassay described above is the only reliable assay which is described in the monograph for BoNT/A in the European pharmacopeia. However, there is a need for reliable assays for measuring neurotoxin activity which avoid the drawbacks of the assays described in the art.

Therefore, the technical problem underlying the present invention could be seen in the provision of means and methods for complying with the aforementioned needs. The technical problem is solved by the embodiments characterized in the claims and herein described below.

The present invention relates to a polynucleotide encoding a fusion polypeptide comprising (i) a transcription factor domain and (ii) a cytoplasmic retention domain, separated by a linker comprising a neurotoxin cleavage site.

The term “polynucleotide” as used herein refers to single- or double-stranded DNA molecules as well as to RNA molecules. Encompassed by the said term is genomic DNA, cDNA, hnRNA, mRNA as well as all naturally occurring or artificially modified derivatives of such molecular species. The polynucleotide may be in an aspect a linear or circular molecule. Moreover, in addition to the nucleic acid sequences encoding the fusion polypeptide of the present invention, a polynucleotide of the present invention may comprise additional sequences required for proper transcription and/or translation such as 5′- or 3′-UTR sequences. In light of the degeneracy of the genetic code, optimized codons may be used in the nucleic acid sequences encoding the fusion polypeptide of the present invention. Thereby, optimal expression in, e.g., a host cell of the present invention can be achieved. In another aspect, the said polynucleotide comprises a nucleic acid sequence as shown in SEQ ID NO: 1, 3, 5, 7, 9, 11, 13, 15, 17, 19, 21, 23, 25, 27, or 29. Moreover, encompassed is in an aspect a polynucleotide comprising a nucleic acid sequence encoding an amino acid sequence as shown in SEQ ID NO: 2, 4, 6, 8, 10, 12, 14, 16, 18, 20, 22, 24, 26, 28, 30 or 31.

In another aspect, the said polynucleotide is a variant of the aforementioned polynucleotides comprising one or more nucleotide substitutions, deletions and/or additions which in still another aspect may result in a fusion polypeptide having one or more amino acid substitutions, deletions and/or additions. Moreover, a variant polynucleotide of the invention shall in another aspect comprise a nucleic acid sequence variant being at least 40%, at least 50%, at least 60%, at least 70%, at least 75%, at least 80%, at least 85%, at least 90%, at least 95%, at least 98% or at least 99% identical to the nucleic acid sequence as shown in any one of SEQ ID NO: 1, 3, 5, 7, 9, 11, 13, 15, 17, 19, 21, 23, 25, 27, or 29 or a nucleic acid sequence variant which encodes an amino acid sequence being at least 40%, at least 50%, at least 60%, at least 70%, at least 75%, at least 80%, at least 85%, at least 90%, at least 95%, at least 98% or at least 99% identical to the amino acid sequence as shown in any one of SEQ ID NO: 2, 4, 6, 8, 10, 12, 14, 16, 18, 20, 22, 24, 26, 28, 30 or 31. The term “identical” as used herein refers to sequence identity characterized by determining the number of identical amino acids between two nucleic acid sequences or amino acid sequences wherein the sequences are aligned so that the highest order match is obtained. It can be calculated using published techniques or methods codified in computer programs such as, for example, BLASTP, BLASTN or FASTA (Altschul 1990, J. Mol. Biol. 215, 403). The percent sequence identity values are, in one aspect, calculated over the entire nucleotide or amino acid sequence. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (Higgins 1989, CABIOS 5, 151) or the programs Gap and BestFit (Needleman 1970, J. Mol. Biol. 48; 443; Smith 1981, Adv. Appl. Math. 2, 482), which are part of the GCG software packet (Genetics Computer Group 1991, 575 Science Drive, Madison, Wisconsin, USA 53711), may be used. The sequence identity values recited above in percent (%) are to be determined, in another aspect of the invention, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments. In an aspect, each of the aforementioned variant polynucleotides encodes a fusion polypeptide retaining one or more and, in another aspect, all of the biological properties of the respective fusion polypeptide of the invention. Preferably, the variant polynucleotide encodes a fusion polypeptide comprising (i) a transcription factor domain and (ii) a cytoplasmic retention domain, separated by a linker comprising a neurotoxin cleavage site.

The term “transcription factor” as used herein means a protein required to initiate or regulate transcription in eukaryotes. A transcription factor is a protein that binds to specific DNA sequences, thereby controlling the flow of genetic information from DNA to mRNA, i.e. the transcription. The DNA sequence that a transcription factor binds to is called a transcription factor-binding site or response element. An “enhancer” as used herein is a short region of DNA that can increase transcription of genes, whereas a “silencer” is a DNA sequence capable of binding transcription regulation factors termed repressors. Transcription factors perform this function alone or with other proteins in a complex, by promoting as an activator or blocking as a repressor the recruitment of RNA polymerase to specific genes. The term “domain” as used herein denotes a protein domain which is a part of protein sequence and structure that can evolve, function, and exist independently of the rest of the protein chain. Each domain forms a compact three-dimensional structure and often can be independently stable and folded. Many proteins, including transcription factors, consist of several structural domains. The term “transcription factor domain” as used herein comprises a transcription factor of a protein domain of a transcription factor. Transcription factors are modular in structure and contain several domains, including, but not limited to, a DNA binding domain, a transactivator domain or silencer domain and an optional signal sensing domain, e.g. a ligand binding domain. The portion of the transcription factor that binds to DNA is called DNA binding domain. The DNA binding domain attaches to specific sequences of DNA such as enhancers or promoters. Transcription factors interact with their binding sites using a combination of electrostatic and van der Waals forces. Due to the nature of these chemical interactions, most transcription factors bind DNA in a sequence specific manner. However, not all bases in the transcription factor-binding site may actually interact with the transcription factor. In addition, some of these interactions may be weaker than others. Thus, transcription factors do not bind just one sequence but are capable of binding a subset of closely related sequences, each with a different strength of interaction. The transactivating domain, i.e. the transactivator or silencer domain, contains binding sites for other proteins such as transcription co-regulators. Transactivator or silencer domains are named after their amino acid composition. These amino acids are either essential for the activity or simply the most abundant in the transactivator or silencer domain. A transcription factor regulates transcription by controlling the rate of gene transcription, for example, by activating or inhibiting RNA polymerase binding to DNA. The transactivating domain may increase the rate of gene transcription or decrease the rate of gene transcription. In the former case, the transactivating domain is called a transactivator domain, in the latter case, the transactivating domain is called a silencer domain. The signal sensing domain senses external signals and, in response, transmit these signals to the rest of the transcription complex, resulting in up- or down-regulation of gene expression. A transcription factor as used herein may be, for example, a tetracycline dependent transactivator (tet-repressor—VP 16), steroid hormone receptors, NOTCH, NFKB, p53, NFAT, MLL, E2A, HSF1, NF-IL6, STAT, R-SMAD, GAL4, or SP1. It is envisaged that the transcription factor as used herein may in some aspects comprise a nuclear localization sequence. Nuclear localization sequences or signals may be either signal sequences or signal patches. The nuclear localization signals direct proteins to the nucleus of the cell.

The term “cytoplasmic retention domain” as used herein denotes a protein domain which mediates a cytoplasmic localization of the fusion polypeptide encoded by the polynucleotide of the invention, prior to cleavage of the fusion protein by the neurotoxin. The cytoplasmic retention domain as used herein can be, for instance, a transmembrane protein or a transmembrane spanning domain of a transmembrane protein. For example, the transmembrane protein may be plasmalemmal neurotransmitter transporters, ion channels, G-protein coupled receptors, or membrane receptors. The cytoplasmic retention domain can also be a membrane-anchored protein or a membrane anchor domain of a membrane-anchored protein. Such a membrane-anchored protein may be, for example, SNAP-25, Syntaxin, synaptoprevin, synaptotagmin, vesicle associated membrane proteins (VAMPs), synaptic vesicle glycoproteins (SV2), high affinity choline transporters, Neurexins, voltage-gated calcium channels, acetylcholinesterase, or NOTCH. The cytoplasmic retention domain as used herein may also be a globular protein or a cytoskeleton anchor protein which is too big to be able to pass the pores of the nucleus. After expression of the fusion polypeptide of the invention, the transcription factor domain is immobilized, for example, to the plasma or vesicle membrane via the cytoplasmic retention domain and is, therefore, not able to translocate to the nucleus. By this way, the expression of the reporter in the nucleus by the transcription factor is avoided. Only the cleavage of the fusion protein of the invention within the linker by the neurotoxin light chain of the Botulinum neurotoxin releases the transcription factor which subsequently translocates to the cell nucleus, thereby initiating the expression of the reporter gene.

The term “linker” as used herein denotes a polylinker which is a short segment of amino acid sequence comprising a neurotoxin cleavage site. Suitable linker sequences encoded by a corresponding DNA sequence are described, for example, in Schiavo G, Matteoli M, Montecucco C: Neurotoxins affecting neuroexocytosis. Physiol. Rev. 2000, 80:717-66.

The term “neurotoxin cleavage site” as used herein refers to a cleavage site which is recognized and cleaved by the endogenous protease of a neurotoxin polypeptide. Cleavage site which are recognized by the neurotoxin proteases are well known in the art; see, e.g., EP 1 926 744 B1. In principle, a neurotoxin cleavage site can be a cleavage site which naturally occurs in a substrate or which is an artificially designed cleavage site recognized and cleaved by the neurotoxin polypeptides protease. It will be understood that the properties of the neurotoxin cleavage site govern the kind of neurotoxin which can activate the fusion polypeptide of the present invention. Neurotoxin polypeptides referred to herein, in an aspect, encompass BoNT/A, BoNT/B, BoNT/C1, BoNT/D, BoNT/E, BoNT/G, BoNT/F or TeNT all of which are well known in the art. For example, if a neurotoxin cleavage site is used which is specifically recognized and cleaved by BoNT/A, only the BoNT/A protease will be capable of activating the fusion polypeptide of the present invention, whereas if a neurotoxin cleavage site is used which is specifically recognized and cleaved by BoNT/E, only the BoNT/E protease will be capable of activating the fusion polypeptide of the present invention. In an aspect of the fusion polypeptide of the invention, the neurotoxin cleavage site is cleaved by mature BoNTs. In yet another aspect, it is cleaved by muteins of BoNTs, in an aspect, by muteins comprising or consisting of the BoNT light chain exhibiting the BoNT protease activity.

A neurotoxin cleavage site recognized and cleaved by the BoNT/A protease, in an aspect of the invention, is derived from a protein that is sensitive to cleavage by BoNT/A. In an aspect, such a protein is human SNAP25A or SNAP25B or a homolog, paralog or ortholog thereof from rat, mouse, bovine, Danio, Carassius, Xenopus, Torpedo, Strongylocentrotus, Loligo, Lymnaea or Aplysia. Suitable cleavage sites derived from said proteins are disclosed in EP 1 926 744 B1.

A neurotoxin cleavage site recognized and cleaved by the BoNT/B protease, in an aspect of the invention, is derived from a protein that is sensitive to cleavage by BoNT/B. In an aspect, such a protein is human or mouse VAMP-1, VAMP-2 and VAMP-3/cellubrevin, bovine VAMP-2, rat VAMP-2 or VAMP-3, chicken VAMP-1, VAMP-2 or VAMP-3, Torpedo VAMP-1, Strongylocentrotus VAMP, Drosophila sybA, synB, synC, synD, or syn, Hirudo VAMP, Xenopus VAMP-2 or VAMP-3, Danio VAMP-1 or VAMP-2, Loligo VAMP, Lymnaea VAMP, Aplysia VAMP or Caenorhabditis SNB1-like or any ortholog, paralog or homolog thereof. Suitable cleavage sites derived from said proteins are disclosed in EP 1 926 744 B1.

A neurotoxin cleavage site recognized and cleaved by the BoNT/C1 protease, in an aspect of the invention, is derived from a protein that is sensitive to cleavage by BoNT/C1. In an aspect, such a protein is human and mouse Syntaxin 1A, Syntaxin 1B1, Syntaxin 2-1, Syntaxin 2-2, Syntaxin 2-3, Syntaxin 3A or Syntaxin 1B2, bovine or rat Syntaxin 1A, Syntaxin 1B1 or Syntaxin 1B2, rat Syntaxin 2 or Rat syntaxin 3, mouse Syntaxin 1A, Syntaxin 1B1, Syntaxin 1B2, Syntaxin 2, Syntaxin 3A, Syntaxin 3B or Syntaxin 3C, chicken Syntaxin 1A or Syntaxin 2; Xenopus Syntaxin 1A or Syntaxin 1B, Danio Syntaxin 1A, Syntaxin 1B or Syntaxin 3, Torpedo Syntaxin 1A or Syntaxin 1B, Strongylocentrotus Syntaxin 1A or Syntaxin 1B, Drosophila Syntaxin 1A or Syntaxin 1B, Hirudo Syntaxin 1A or Syntaxin 1B, Loligo Syntaxin 1A or Syntaxin 1B, Lymnaea Syntaxin 1A or Syntaxin 1B or any ortholog, paralog or homolog thereof. Suitable cleavage sites derived from said proteins are disclosed in EP 1 926 744 B1.

A neurotoxin cleavage site recognized and cleaved by the BoNT/D protease, in an aspect of the invention, is derived from a protein that is sensitive to cleavage by BoNT/D. In an aspect, such a protein is human or mouse VAMP-1, VAMP-2 and VAMP-3/cellubrevin, bovine VAMP-2, rat VAMP-2 or VAMP-3, chicken VAMP-1, VAMP-2 or VAMP-3, Torpedo VAMP-1, Strongylocentrotus VAMP, Drosophila sybA, synB, synC, synD, or syn, Hirudo VAMP, Xenopus VAMP-2 or VAMP-3, Danio VAMP-1 or VAMP-2, Loligo VAMP, Lymnaea VAMP, Aplysia VAMP or Caenorhabditis SNB1-like or any ortholog, paralog or homolog thereof. Suitable cleavage sites derived from said proteins are disclosed in EP 1 926 744 B1.

A neurotoxin cleavage site recognized and cleaved by the BoNT/E protease, in an aspect of the invention, is derived from a protein that is sensitive to cleavage by BoNT/E. In an aspect, such a protein is, such a protein is human SNAP-25A or B or a homolog, paralog or ortholog thereof from rat, mouse, bovine, Danio, Carassius, Xenopus, Torpedo, Strongylocentrotus, Loligo, Lymnaea or Aplysia. Suitable cleavage sites derived from said proteins are disclosed in EP 1 926 744 B1.

A neurotoxin cleavage site recognized and cleaved by the BoNT/F protease, in an aspect of the invention, is derived from a protein that is sensitive to cleavage by BoNT/F. In an aspect, such a protein is, such a protein is human or mouse VAMP-1, VAMP-2 and VAMP-3/cellubrevin, bovine VAMP-2, rat VAMP-2 or VAMP-3, chicken VAMP-1, VAMP-2 or VAMP-3, Torpedo VAMP-1, Strongylocentrotus VAMP, Drosophila sybA, synB, synC, synD, or syn, Hirudo VAMP, Xenopus VAMP-2 or VAMP-3, Danio VAMP-1 or VAMP-2, Loligo VAMP, Lymnaea VAMP, Aplysia VAMP or Caenorhabditis SNB1-like or any ortholog, paralog or homolog thereof. Suitable cleavage sites derived from said proteins are disclosed in EP 1 926 744 B1.

A neurotoxin cleavage site recognized and cleaved by the BoNT/G protease, in an aspect of the invention, is derived from a protein that is sensitive to cleavage by BoNT/G. In an aspect, such a protein is, such a protein is human or mouse VAMP-1, VAMP-2 and VAMP-3/cellubrevin, bovine VAMP-2, rat VAMP-2 or VAMP-3, chicken VAMP-1, VAMP-2 or VAMP-3, Torpedo VAMP-1, Strongylocentrotus VAMP, Drosophila sybA, synB, synC, synD, or syn, Hirudo VAMP, Xenopus VAMP-2 or VAMP-3, Danio VAMP-1 or VAMP-2, Loligo VAMP, Lymnaea VAMP, Aplysia VAMP or Caenorhabditis SNB1-like or any ortholog, paralog or homolog thereof. Suitable cleavage sites derived from said proteins are disclosed in EP 1 926 744 B1.

A neurotoxin cleavage site recognized and cleaved by the TeNT protease, in an aspect of the invention, is derived from a protein that is sensitive to cleavage by TeNT. In an aspect, such a protein is human or mouse VAMP-1, VAMP-2 and VAMP-3/cellubrevin, bovine VAMP-2, rat VAMP-2 or VAMP-3, chicken VAMP-1, VAMP-2 or VAMP-3, Torpedo VAMP-1, Strongylocentrotus VAMP, Drosophila sybA, synB, synC, synD, or syn, Hirudo VAMP, Xenopus VAMP-2 or VAMP-3, Danio VAMP-1 or VAMP-2, Loligo VAMP, Lymnaea VAMP, Aplysia VAMP or Caenorhabditis SNB1-like or any ortholog, paralog or homolog thereof. Suitable cleavage sites derived from said proteins are disclosed in EP 1 926 744 B1.

The term “separated by a linker” in context of the polynucleotide or the fusion polypeptide of the invention means that a linker comprising a neurotoxin cleavage site is located between the transcription factor domain and the cytoplasmic retention domain, as set forth in more detail below.

The term “biologically active” used herein in context with a neurotoxin refers to a mature neurotoxin polypeptide exhibiting essentially the biological properties specified herein, i.e. being capable of (a) receptor binding, (b) internalization, (c) translocation across the endosomal membrane into the cytosol, and/or (d) endoproteolytic cleavage of proteins involved in synaptic vesicle membrane fusion. Preferably, the biologically activity is the proteolytic activity of neurotoxins.

It is to be understood that the definitions and explanations of the terms made above apply mutatis mutandis for all aspects described in the specification in the following except as otherwise indicated.

The means and methods of the invention are based on the proteolytic release of a transcription factor domain from a fusion polypeptide by the Botulinum neurotoxin in cells, preferably in neuronal cells as further defined elsewhere herein. The transcription factor domain can in one aspect of the polynucleotide or fusion polypeptide of the invention be fused to a cytoplasmic retention domain via a linker comprising a neurotoxin cleavage site. After expression of said fusion polypeptide, the transcription factor domain is first immobilized to the cytoplasm. This can be achieved by using suitable cytoplasmic retention signals in the polynucleotide or fusion polypeptide of the invention, such as a transmembrane protein or a transmembrane spanning domain of a transmembrane protein, a membrane-anchored protein or a membrane anchor domain of a membrane-anchored protein, or a globular protein or cytoskeleton anchor protein. The cytoplasmic retention signal prevents the transcription factor domain from translocating to the cell nucleus. Only the proteolytic activity of a Botulinum neurotoxin releases the transcription factor domain from the fusion polypeptide upon cleavage of the fusion protein within the linker comprising the neurotoxin cleavage site. This results in the translocation of the transcription factor domain to the nucleus where it is able to initiate the expression of a reporter protein, such as luciferase, alkaline phosphatase, or horseradish peroxidase. The presence and/or the amount of the expressed reporter protein can be analyzed by assays well described in the art, for instance by an enzymatic test (Miraglia L J, King F J, Damoiseaux R: Seeing the light: luminescent reporter gene assays. Comb. Chem. High Throughput Screen 2011, 14:648-57; Fan F, Wood KV: Bioluminescent assays for high-throughput screening. Assay Drug Dev. Technol. 2007, 5:127-36). The amount of the expressed reporter is directly proportional to the proteolytic activity of the neurotoxin light chain in the cytoplasm and, thus, also proportional to the biological activity of Botulinum neurotoxin. By comparison of the results of such an assay with a reference Botulinum neurotoxin with a defined biological activity, the biological activity of an unknown Botulinum neurotoxin in a sample can be determined.

Advantageously, the polynucleotide and the fusion polypeptide of the invention allow for the determination of the protease activity of a given neurotoxin polypeptide in a cell culture system. Thus, expensive and ethically unnecessary animal testing can be avoided or reduced thanks to the present invention. Moreover, the polynucleotide and the fusion polypeptide of the invention can be applied in automated high throughput screening assays. The polynucleotide and the fusion polypeptide of the invention also allow for establishing assays in cell lines, such as, e.g., neuroblastoma cell lines. Accordingly, the use of primary neurons as required in other cell based neurotoxin assays can be avoided. This is another advantage of the methods and means of the invention since the preparation of primary neurons is cumbersome and inefficient. In addition, by using the means and methods described therein, the sensitivity and flow rate of cell based assays for the determination of the biological activity of Botulinum neurotoxins could be significantly increased. Therefore, the present invention provides improved assays for testing the biological activity of Botulinum neurotoxins.

In an aspect of the polynucleotide of the invention, the cytoplasmic retention domain is a transmembrane protein or transmembrane spanning domain of a transmembrane protein. By using suitable cytoplasmic retention domains, the fusion protein of the invention is transported to or retained in the compartment of the cell, where the neurotoxin light chain is biologically active, i.e. the cytoplasm. Put in other words:

After expression of said fusion polypeptide, the transcription factor domain is, prior to the activation by neurotoxin cleavage, immobilized to the cytoplasm. Such a cytoplasmic retention domain is in this aspect of the polynucleotide encoding the fusion polypeptide of the invention a transmembrane protein or transmembrane spanning domain of a transmembrane protein.

In another aspect of the polynucleotide of the invention, the transmembrane protein is selected from the group consisting of: plasmalemmal neurotransmitter transporters, ion channels, G-protein coupled receptors and membrane receptors.

In a further aspect of the polynucleotide of the invention, the cytoplasmic retention domain is a membrane-anchored protein or a membrane anchor domain of a membrane-anchored protein. In one aspect of the polynucleotide of the invention, the membrane-anchored protein is selected from the group consisting of: SNAP-25, Syntaxin, synaptoprevin, synaptotagmin, vesicle associated membrane proteins (VAMPs), synaptic vesicle glycoproteins (SV2), high affinity choline transporters, Neurexins, voltage-gated calcium channels, acetylcholinesterase, and NOTCH. In the following, the corresponding accession number of the respective protein is indicated: human SNAP-25 P60880, human Syntaxin-1A Q16623, Syntaxin-1B P61266, Syntaxin-2 P32856, Syntaxin-3 Q13277, Syntaxin-4 Q12846, Syntaxin-5 Q13190, Syntaxin-6 043752, Syntaxin-7 Q15400, Syntaxin-8 Q9UNKO, Syntaxin-10 O60499, Syntaxin-11 075558, Syntaxin-12 Q86Y82, Syntaxin-16 O14662, Syntaxin-17 P56962, Syntaxin-18 Q9P2W9, Syntaxin-19 Q8N4C7; human Synaptobrevin-1 P23763, Synaptobrevin-2 P63027, Synaptobrevin-3 Q15836; human synaptotagmin: Synaptotagmin-1 P21579, Synaptotagmin-2 Q8N9I0, Synaptotagmin-3 Q9BQG1, Synaptotagmin-4 Q9H2B2, Synaptotagmin-5 000445, Synaptotagmin-6 Q5T7P8, Synaptotagmin-8 Q8NBV8, Synaptotagmin-9 Q86SS6, Synaptotagmin-10 Q6XYQ8, Synaptotagmin-11 Q9BT88, Synaptotagmin-12 Q8IV01, Synaptotagmin-13 Q7L8C5, Synaptotagmin-14 Q8NB59, Synaptotagmin-15 Q9BQS2, Synaptotagmin-16 Q17RD7, Synaptotagmin-17 Q9BSW7, human vesicle associated membrane proteins (VAMPs): Vesicle-associated membrane protein 1 P23763, Vesicle-associated membrane protein 2 P63027, Vesicle-associated membrane protein 3 Q15836, Vesicle-associated membrane protein 4 O75379, Vesicle-associated membrane protein 5 O95183, Vesicle-associated membrane protein 7 P51809, Vesicle-associated membrane protein 8 Q9BV40; of synaptic vesicle glycoproteins (SV2): Synaptic vesicle glycoprotein 2A Q7L0J3, Synaptic vesicle glycoprotein 2B Q7L112, Synaptic vesicle glycoprotein 2C Q496J9; human high affinity choline transporter Q9GZV3; human Neurexins: Neurexin-1-alpha Q9ULB1, Neurexin-1-beta P58400, Neurexin-2-alpha Q9P2S2, Neurexin-2-beta P58401, of Neurexin-3-alpha Q9Y4C0, Neurexin-3-beta Q9HDB5; human voltage-gated calcium channels: Voltage-dependent calcium channel subunit alpha-2/delta-1 P54289, Voltage-dependent calcium channel subunit Q9NY47, Voltage-dependent calcium channel subunit Q8IZS8, Voltage-dependent calcium channel subunit Q7Z3S7, Voltage-dependent P/Q-type calcium channel subunit O00555, Voltage-dependent N-type calcium channel subunit Q00975, Voltage-dependent L-type calcium channel subunit Q13936, Voltage-dependent L-type calcium channel subunit Q01668, Voltage-dependent R-type calcium channel subunit Q15878, Voltage-dependent L-type calcium channel subunit O60840, Voltage-dependent T-type calcium channel subunit O43497, Voltage-dependent T-type calcium channel subunit O95180, Voltage-dependent T-type calcium channel subunit Q9P0X4, Voltage-dependent L-type calcium channel subunit Q13698, Voltage-dependent L-type calcium channel subunit Q02641, Voltage-dependent L-type calcium channel subunit Q08289, Voltage-dependent L-type calcium channel subunit P54284, Voltage-dependent L-type calcium channel subunit O00305, Voltage-dependent calcium channel gamma-1 subunit Q06432, Voltage-dependent calcium channel gamma-2 subunit Q9Y698,

Voltage-dependent calcium channel gamma-3 subunit O60359, Voltage-dependent calcium channel gamma-4 subunit Q9UBN1, Voltage-dependent calcium channel gamma-5 subunit Q9UF02, Voltage-dependent calcium channel gamma-6 subunit Q9BXT2, Voltage-dependent calcium channel gamma-7 subunit P62955, Voltage-dependent calcium channel gamma-8 subunit Q8WXS5, Voltage-dependent calcium channel gamma-like subunit Q8WXS4, Voltage gated calcium channel alpha 1F subunit F5CIQ9, Sodium leak channel non-selective protein Q8IZFO, Voltage-gated calcium channel beta 2 subunit Q5QJ99, Voltage-gated calcium channel beta 2 subunit Q5QJA0, Voltage-gated L-type calcium channel Cav1.2 Q5V9X8, Voltage-gated L-type calcium channel Cav1.2 Q5V9X9, Voltage-gated calcium channel beta 2 subunit Q6TME0, Voltage-gated calcium channel beta 2 subunit Q6TME1, Voltage-gated calcium channel beta 2 subunit Q6TME2, Voltage-gated calcium channel beta 2 subunit Q6TME3, Voltage-gated calcium channel beta 1 subunit Q6TME4, Voltage-gated calcium channel alpha 1 subunit Q71UT1, Voltage-gated calcium channel alpha 1E subunit Q9UN68; human acetylcholinesterase P22303; and human NOTCH: Neurogenic locus notch homolog protein 1 P46531, Neurogenic locus notch homolog protein 2 Q04721, Neurogenic locus notch homolog protein 3 Q9UM47, Neurogenic locus notch homolog protein 4 Q99466.

In this aspect of the polynucleotide of the invention, it is advantageous to select proteins which are localized at or in the pre-synaptic membrane. It is to be understood that basically all proteins can be used which by membrane-anchor or interaction with membrane proteins are localized at the membrane, e.g. plasma membrane or vesicle membrane. Such proteins are preferably present at the compartment where the neurotoxin light chain exerts its biological activity, i.e. in the cytoplasm of a cell, e.g. a neuronal cell. The biological activity is composed of the binding to the neurotoxin receptors, the translocation of the neurotoxin light chain into the cytosol of the target cell and the proteolytic activity of the neurotoxin mediated by the light chain of the neurotoxin.

In a still further aspect of the polynucleotide of the invention, the cytoplasmic retention domain is selected from a globular protein or a cytoskeleton anchor protein. In this aspect, the transcription factor is fused to a large globular protein or cytoskeleton anchor protein which is big enough to prevent the fusion polypeptide from passing the pores of the nucleus. By this way, the transport of the fusion protein (comprising the transcription activator domain) through the pores of the nucleus can be avoided. It is envisaged that a globular protein or a cytoskeleton anchor protein can be used alone or fused to other proteins or protein domains, such as exportin binding domains or IKB binding domains. The invention has been exemplified for the leucine-rich nuclear export signal (NES): (LxxxLxxLxL; SEQ ID NO: 32), with “x” representing any naturally occurring amino acid.

In another aspect of the polynucleotide of the invention, the transcription factor domain comprises a transcription factor comprising a DNA binding domain and a transactivator or silencer domain. In a further aspect, the transcription factor comprises, in addition to a DNA binding domain and a transactivator or silencer domain, a nuclear localization sequence. For example, in some aspects, the silencer or repressor (e.g. REST) can be positioned opposite to the transactivator domain so that the cleavage site intervenes silencer/repressor and transactivator domain. This results in an additional decrease in basal expression of the reporter. The nuclear localization signals direct proteins to the nucleus of the cell. It is to be understood that the transcription factor as used herein is able to modulate the expression of a reporter gene, i.e. the transcription factor is capable of increasing or decreasing the expression of a reporter gene.

In a still further aspect of the polynucleotide of the invention, the transcription factor is selected from the group consisting of: tetracycline dependent transactivator (tet-repressor—VP 16), steroid hormone receptors, NOTCH, NFKB, p53, NFAT, MLL, E2A, HSF1, NF-IL6, STAT, R-SMAD, GAL4, and SP1. The amino acid sequence for the tetracycline dependent transactivator is shown, e.g., in SEQ ID NO: 33 or 34:

(SEQ ID NO: 33)
“SRLDKSKVINSALELLNGVGIEGLTTRKLAQKLGVEQPTLYWHVKN
KRALLDALPIEMLDRHHTHSCPLEGESWQDFLRNNAKSYRCALLSHR
DGAKVHLGTRPTEKQYETLENQLAFLCQQGFSLENALYALSAVGHFT
LGCVLEEQEHQVAKEERETPTTDSMPPLLKQAIELFDRQGAEPAFLF
GLELIICGLEKQLKCESGGPTDALDDFDLDMLPADALDDFDLDMLPA
DALDDFDLDMLPG”
(SEQ ID NO: 34)
“SRLDKSKVINSALELLNEVGIEGLTTRKLAQKLGVEQPTLYWHVKN
KRALLDALAIEMLDRHHTHFCPLEGESWQDFLRNNAKSFRCALLSHR
DGAKVHLGTRPTEKQYETLENQLAFLCQQGFSLENALYALSAVGHFT
LGCVLEDQEHQVAKEERETPTTDSMPPLLRQAIELFDHQGAEPAFLF
GLELIICGLEKQLKCESGSAYSRARTKNNYGSTIEGLLDLPDDDAPE
EAGLAAPRLSFLPAGHTRRLSTAPPTDVSLGDELHLDGEDVAMAHAD
ALDDFDLDMLGDGDSPGPGFTPHDSAPYGALDMADFEFEQMFTDALG
IDEYGG”.

The amino acid sequence of the tet-repressor is shown under accession number P04483. The amino acid sequence of VP16 is shown under accession number P06492. The corresponding accession numbers for steroid hormone or other nuclear receptors are: Androgen receptor P10275, Estrogen receptor P03372, Glucocorticoid receptor P04150, Mineralocorticoid receptor P08235, Progesterone receptor P06401, Prolactin receptor P16471, Retinoic acid receptor alpha P10276, Retinoic acid receptor gamma P13631, Retinoic acid receptor RXR-alpha P19793, Steroid hormone receptor ERR1 P11474, Steroid hormone receptor ERR2 O95718, Thyroid hormone receptor alpha P10827, Thyroid hormone receptor beta P10828. The corresponding accession numbers for NOTCH proteins are: Neurogenic locus notch homolog protein 1 P46531, Neurogenic locus notch homolog protein 2 Q04721, Neurogenic locus notch homolog protein 3 Q9UM47, Neurogenic locus notch homolog protein 4 Q99466. The corresponding accession number for NFKB is Q04206; for p53 PO4637; for NFAT: Nuclear factor of activated T-cells 1 095644, Nuclear factor of activated T-cells 2 Q13469, Nuclear factor of activated T-cells 3 Q12968, Nuclear factor of activated T-cells 4 Q14934, Nuclear factor of activated T-cells 5 094916; for E2A (Transcription factor E2-alpha) P15923; for HSF1 Q00613; for NF-IL6 P17676; for STAT: STAT1_HUMAN P42224, STAT2_HUMAN P52630, STAT3_HUMAN P40763, STAT4_HUMAN Q14765, STA5A_HUMAN P42229, STA5B_HUMAN P51692, STAT6_HUMAN P42226; for R-SMAD: Mothers against decapentaplegic homolog 1 Q15797, Mothers against decapentaplegic homolog 2 Q15796, Mothers against decapentaplegic homolog 3 P84022, Mothers against decapentaplegic homolog 4 Q13485, Mothers against decapentaplegic homolog 5 Q99717, Mothers against decapentaplegic homolog 6 O43541, Mothers against decapentaplegic homolog 7 O15105, Mothers against decapentaplegic homolog 9 O15198; for GAL4, and for SP1 P08047.

The invention also pertains to a vector comprising the polynucleotide of the invention. In an aspect, the said vector is an expression vector.

The term “vector”, preferably, encompasses phage, plasmid, viral or retroviral vectors as well as artificial chromosomes, such as bacterial or yeast artificial chromosomes. Moreover, the term also relates to targeting constructs which allow for random or site-directed integration of the targeting construct into genomic DNA. Such target constructs, preferably, comprise DNA of sufficient length for either homologous or heterologous recombination as described in detail below. The vector encompassing the polynucleotides of the present invention, in an aspect, further comprises selectable markers for propagation and/or selection in a host. The vector may be incorporated into a host cell by various techniques well known in the art. For example, a plasmid vector can be introduced in a precipitate such as a calcium phosphate precipitate or rubidium chloride precipitate, or in a complex with a charged lipid or in carbon-based clusters, such as fullerens. Alternatively, a plasmid vector may be introduced by heat shock or electroporation techniques. Should the vector be a virus, it may be packaged in vitro using an appropriate packaging cell line prior to application to host cells. Retroviral vectors may be replication competent or replication defective. In the latter case, viral propagation generally will occur only in complementing host/cells. Moreover, in an aspect of the invention, the polynucleotide is operatively linked to expression control sequences allowing expression in prokaryotic or eukaryotic host cells or isolated fractions thereof in the said vector. Expression of the polynucleotide comprises transcription of the polynucleotide into a translatable mRNA. Regulatory elements ensuring expression in host cells are well known in the art. In an aspect, they comprise regulatory sequences ensuring initiation of transcription and/or poly-A signals ensuring termination of transcription and stabilization of the transcript. Additional regulatory elements may include transcriptional as well as translational enhancers. Possible regulatory elements permitting expression in prokaryotic host cells comprise, e.g., the lac-, trp- or tac- promoter in E. coli, and examples for regulatory elements permitting expression in eukaryotic host cells are the AOX1- or the GAL1-promoter in yeast or the CMV-, SV40-, RSV-promoter (Rous sarcoma virus), CMV-enhancer, SV40-enhancer or a globin intron in mammalian and other animal cells. Other expression systems envisaged by the invention shall permit expression in insect cells, such as polyhedrin promoter based systems.

Moreover, inducible expression control sequences may be used in an expression vector encompassed by the present invention. Inducible expression systems and suitable expression control sequences are well known in the art. For example, the tetracycline-responsive regulatory system for transcriptional transactivation is described in Zhu Z, Zheng T, Lee C G, Homer R J, Elias J A: Tetracycline-controlled transcriptional regulation systems: advances and application in transgenic animal modeling. Semin. Cell Dev. Biol. 2002, 13:121-8; or Shockett P, Schatz D: Inducible gene expression using an autoregulatory, tetracycline-controlled system. Curr. Protoc. Mol. Biol. 2002, Chapter 16: Unit 16.14. Such inducible vectors may comprise tet or lac operator sequences or sequences inducible by heat shock or other environmental factors are described in the art. For example, two commonly used inducible expression systems are Tet-Off and Tet-On; see Bujard and Gossen, Proc. Natl. Acad. Sci. U.S.A. 89 (12): 5547-51. They consist of a fusion of the Tet repressor and a VP16 activation domain to create a transcriptional activator protein (transactivator) rather than a repressor. Gene expression is activated as a result of binding of the Tet-Off or Tet-On protein to tetracycline response elements (TREs) located within an inducible promoter. The difference relates to their respective response to doxycycline (Dox), a more stable tetracycline analogue: Tet-Off activates expression in the absence of Dox, whereas Tet-On activates in the presence of Dox. Suitable vectors are commercially available. For example, the Tet-On 3G vector set by Clontech can be used to create tightly regulated and highly responsive tetracycline (Tet)-inducible mammalian expression systems that are turned on by the addition of doxycycline to the culture medium. The pCMV-Tet3G vector expresses Tet-On 3G, a tetracycline-controlled transactivator that exhibits high activity in the presence of the inducer doxycycline, and exceptionally low activity in its absence. Tet-On 3G results from the fusion of amino acids 1-207 of a mutant Tet repressor (TetR) to 39 amino acids that form three minimal “F”-type transcriptional activation domains from the herpes simplex virus VP16 protein. Constitutive expression of Tet-On 3G is driven by the human cytomegalovirus immediate early promoter (P_{CMV IE}). Further, an EF1alpha version is available for cell lines in which the CMV promoter is silenced. For further detailed information see Clontech catalogue number 631163 and references cited therein. Beside elements which are responsible for the initiation of transcription such regulatory elements may also comprise transcription termination signals, such as the SV40-poly-A site or the tk-poly-A site, downstream of the polynucleotide. In this context, suitable expression vectors are known in the art such as Okayama-Berg cDNA expression vector pcDV1 (Pharmacia), pBluescript (Stratagene), pCDM8, pRc/CMV, pcDNA1, pcDNA3, pcDNA3.1 (Invitrogen) or pSPORT1 (Invitrogen) or baculovirus-derived vectors. Preferably, said vector is an expression vector and a gene transfer or targeting vector. Expression vectors derived from viruses such as retroviruses, vaccinia virus, adeno-associated virus, herpes viruses, or bovine papilloma virus, may be used for delivery of the polynucleotides or vector of the invention into targeted cell population. Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994).

Furthermore, the invention relates to a fusion polypeptide encoded by the polynucleotide of the invention. The term “fusion polypeptide” as used herein denotes a fusion polypeptide comprising (i) a transcription factor domain and (ii) a cytoplasmic retention domain. The transcription factor domain and the cytoplasmic retention domain are separated by a linker comprising a neurotoxin cleavage site. Proteolytic cleavage in the linker occurs at the neurotoxin cleavage site in a manner such that the linker is cleaved (at least) once. The linker may comprise one or more neurotoxin cleavage site(s). The term “separated by a linker” in context of the polynucleotide encoding the fusion polypeptide or the fusion polypeptide of the invention means that a linker comprising a neurotoxin cleavage site is located between the transcription factor domain and the cytoplasmic retention domain. Accordingly, the domain arrangement in the fusion polypeptide can be from the N-terminus transcription factor domain-linker-cytoplasmic retention domain to the C-terminus or from the N-terminus cytoplasmic retention domain-linker-transcription factor domain to the C-terminus. The term “fusion polypeptide” as used herein encompasses isolated or essentially purified polypeptides being essentially free of other host cell polypeptides. The term, in another aspect, includes polypeptide preparations comprising the fusion polypeptide of the present invention and other proteins in addition. Moreover, the term includes, in an aspect, chemically modified polypeptides. Such modifications may be artificial modifications or naturally occurring modifications. The fusion polypeptide of the present invention shall have the biological properties referred to above. Moreover, encompassed is in an aspect a fusion polypeptide comprising an amino acid sequence as shown in SEQ ID NO: 2, 4, 6, 8, 10, 12, 14, 16, 18, 20, 22, 24, 26, 28, 30 or 31. The polypeptide of the invention, in an aspect, can be manufactured by chemical synthesis or recombinant molecular biology techniques well known for the skilled artisan. In an aspect, such a method of manufacturing the fusion polypeptide of the invention comprises (a) culturing the host cell of the present invention described elsewhere herein in more detail and (b) obtaining from the said host cell the fusion polypeptide of the present invention. In an aspect of this method, the polypeptide can be obtained by conventional purification techniques from a host cell lysate including affinity chromatography, ion exchange chromatography, size exclusion chromatography and/or preparative gel electrophoresis. In an aspect of the fusion polypeptide of the invention, the fusion polypeptide comprises a purification tag.

The term “purification tag” refers to an amino acid sequence which allows for purification of the polypeptide comprising it. This can be achieved in an aspect due to the presence of individual amino acids, peptide sequences or three dimensional peptide structures which are capable of physically or chemically interacting with the purification matrix. Suitable tags include affinity tags and epitope tags. In this context, it is well known that certain amino acids are capable of forming complexes with defined matrices to be used for purification, such as histidines which are capable of physico-chemically interacting with metal ions such as nickel or cobalt embedded in a suitable matrix such as sepharose or agarose. Further peptide sequences or three dimensional structures can be found in chitin binding proteins (CBP), maltose binding proteins (MBP) or glutathione-S-transferases (GST). In a further aspect, protein-protein interaction based purification matrix/purification tag systems can be used such as streptavidin/biotin systems which are also well known in the art. In yet another aspect, the tag may confer a physic-chemical property to the polypeptide containing it which allows for purification, such as fluorescence. Suitable tags in this context include the well known fluorescent proteins, such as green fluorescent protein (GFP), blue fluorescent protein (BFP) or others. In addition to these tags which have an inherent affinity for some matrices, epitope tags can be applied in another aspect. Such epitope tags consists of short amino acid stretches which constitute a peptide epitope that can be specifically recognized by an antibody. Epitope tags are also well known in the art and include FLAG tags, MYC-tags, or HA-tags. Thus, in an aspect, the purification tag is selected from the group consisting of: His-tag, Myc-tag, FLAG-tag, strep-tag, MBP-tag, NusA tag, GST-tag, streptavidin and avidin.

The invention further pertains to a host cell comprising the polynucleotide, the vector or the fusion polypeptide of the invention.

The term “host cell” as used herein encompasses prokaryotic and eukaryotic host cells, preferably isolated prokaryotic and eukaryotic host cells. Preferably, the host cell is a eukaryotic host cell. A eukaryotic host cell as used herein is a cell of an animal cell line suitable for production of the fusion polypeptide of the invention. The polynucleotide or vector of the invention can be stably integrated into the genome of the host cell or transiently expressed by the host cell. A host cell as referred to herein, thus, encompasses in an aspect, yeast cells, mammalian cells, including human cells, plant cells or insect cells, either as primary cells or as cell lines. Preferably, the host cell is a neuronal cell or cell line or a neuroblastoma cell or cell line.

In one aspect of the host cell of the invention, the host cell is selected from the group consisting of: primary neuronal cells, neuroblastoma cell lines, cell line N1E-115, cell line Neuro2a, cell line SH-SY5Y, cell line PC12, cell line SiMa, cell line MHH-NB-11, cell line SK-N-BE(2) and induced pluripotent stem cells (iPS), preferably human induced pluripotent stem cells (hiPS). Neuroblastoma cell lines can be obtained from ATCC or DSMZ under the following ATCC or DSMZ numbers: cell line N1E-115 CRL-2263, cell line Neuro2a CCL-131, cell line SH-SY5Y CRL-2266, cell line PC12 CRL-1721, cell line SiMa ACC 164 (DSMZ), cell line MHH-NB-11 ACC 157 (DSMZ) and cell line SK-N-BE(2) CRL-2271. Specific clones of the cell line SiMa are furthermore disclosed in WO 2010/105234. Methods of generating iPS cells are described, for example, in Yu et al., Science. 2009 May 8; 324(5928): 797-801. Epub 2009, WO 2011/056971 and WO 2011/025852. In some embodiments, iPS are differentiated into neurons using suitable methods, e.g., those described in WO 2012/135621 and U.S. Patent Applications US 2010/0279403 and US 2010/0216181.

In a still further aspect of the host cell of the invention, the host cell comprises a reporter gene the expression of which is modulated by the transcription factor domain upon cleavage of the fusion polypeptide. In this aspect, the host cell comprises a reporter gene. The reporter gene can be stably integrated into the genome of the host cell or transiently expressed by the host cell. The expression of the reporter gene is modulated by the transcription factor domain only after cleavage by a neurotoxin of the fusion polypeptide of the invention within the linker. Reporter gene assays are well described in the art (Miraglia L J, King F J, Damoiseaux R: Seeing the light: luminescent reporter gene assays. Comb. Chem. High Throughput Screen 2011, 14:648-57; Fan F, Wood K V: Bioluminescent assays for high-throughput screening. Assay Drug Dev. Technol. 2007, 5:127-36). As a reporter gene, for example luciferase, alkaline phosphatase, horseradish peroxidase or fluorescent proteins (GFP, YFP, RFP, CFP etc.) can be used.

The invention pertains additionally to a method for determining neurotoxin activity in a sample comprising the steps of:
(a) contacting a host cell comprising the fusion polypeptide of the invention and a reporter gene the expression of which is modulated by the transcription factor domain upon cleavage of the said fusion polypeptide with a sample suspected to comprise neurotoxin activity; and
(b) measuring the reporter gene activity, whereby neurotoxin activity in the sample is determined.

The generation of a reporter gene assay as described herein facilitates and improves the determination of the biological activity of neurotoxin polypeptides such as BoNT/A. For such assays, the polynucleotides, fusion polypeptides, vectors and host cells of the invention can be utilized. Sequentially, an activator construct and a reporter gene construct are stably transfected into host cells as described herein. After intoxication and cleavage of the neurotoxin polypeptide substrate such as SNAP-25, the transactivator (tTA) comprised in the activator construct is released from the Tet system. The binding of the transactivator to regulatory elements in the reporter gene construct results in the activation of the expression of the reporter gene such as luciferase. The activity of the encoded luciferase enzyme can be detected by commercially available kits, within a few hours. The luminescence mediated by the luciferase in a redox reaction is equivalent to the amount of neurotoxin polypeptide in the sample. A standard curve with known neurotoxin polypeptide concentrations is used as a reference in parallel. As a further reference, a protein measurement can be carried out in order to match the signal by luciferase to the cell number in the sample which may vary from case to case. This method is a routine method known in the art and can be carried out in parallel to the activity assays of the invention described above. As host cells the cells described herein can be used such as, for example, the SIMA cell line (human neuroblastoma). In the present invention, various clones of the SIMA cell line have been generated from a sub-cloned SIMA cell line (SIMA no. 3B4) which comprise the activator construct mST01 (synaptotagmin-SNAP-25-tTA) (SEQ ID NO: 9) and the reporter gene construct pTRE-Luc (luciferase reporter) (Clontech), as shown in the following Example. Reporter genes that can be used for the reporter gene assay as described herein are well known in the art and include, for example, the sequences shown in accession numbers P08659, Q27758, H1AD96, A3KC01, A3KC00, A3KBZ9, A3KBZ8, A3KBZ7, A3KBZ6, A3KBZ5, D5MS63, Q2TNU5, Q5UFR2, Q2VL10, Q6SVE0, B5A991, Q27688, A1Z0H9, Q207B1, Q5USC8, G9M6I8, Q6SVE2, Q9GPF9, or Q6SVE1.

The aforementioned clones which comprise the activator construct mST01 (synaptotagmin-SNAP-25-tTA) (SEQ ID NO: 9) and the reporter gene construct pTRE-Luc (luciferase reporter) (Clontech) carry stably the activator and reporter construct in the genome. Furthermore, parental SIMA cells, i.e. not sub-cloned, human multipotent cells from embryonic tissues or the commercially available P19 cell line (murine embryonic carcinoma) can be used as well for transfection with the mentioned constructs of the invention, either as transient transfection or as stable transfection. Preferably, the transfection is a stable transfection. The, thus, generated novel double-transgenic cell line described above allows for an improved assay for the measurement of the biological activity of neurotoxin polypeptides. As a result, the sensitivity of the assay for the determination of the neurotoxin polypeptide activity could be increased. Moreover, the assays of the invention save time and costs in comparison to assays described in the art. Said assays of the invention provide an alternative to methods for the detection of the biological activity of neutrotoxin polypeptides described in the art, such as for examples Western blotting which is a laborious method requiring up to two man days.

The method of the present invention can be assisted by automation. Specifically, in an aspect, step a) and /or b) may be assisted by robotic devices and automated reader systems for mixing compounds and measuring the reporter gene activity. Suitable systems are known in the art and depend on the type of response to be determined. Moreover, the method may comprise additional steps pertaining to the sample preparation or generation of the fusion polypeptide of the present invention.

The term “contacting” as used herein refers to bringing at least two different compounds in physical proximity as to allow physical and/or chemical interaction of said compounds. In the aforementioned method, a host cell comprising the fusion polypeptide of the invention and a reporter gene the expression of which is modulated by the transcription factor domain upon cleavage of the said fusion polypeptide is contacted with a sample suspected to comprise neurotoxin activity. The contacting shall be for a time and under conditions sufficient to allow cleavage of the neurotoxin cleavage site in the linker of the fusion polypeptide of the invention by the neurotoxin polypeptide comprised by the sample. Contacting as used herein, in an aspect, occurs in a host cell of the present invention containing the fusion polypeptide of the present invention. Thus, in an aspect, said polypeptide is comprised by a host cell and, in an aspect, the host cell of the present invention. The said time and conditions will be dependent on the amount of neurotoxin polypeptide comprised by the sample as well as on the uptake of the neurotoxin polypeptide by the host cell. The person skilled in the art is well aware of which conditions need to be applied dependent on the host cell, kind of sample, and kind of neurotoxin which shall be determined. In this aspect, contacting occurs in a cell system comprising the fusion polypeptide of the invention. The cell system shall allow for measuring the activity of the neurotoxin, upon contacting the system with a sample and, thus, allows for determining the neurotoxin protease activity in said sample. The term “sample” refers to a sample suspected to comprise neurotoxin polypeptide. The sample, in an aspect, is an aqueous solution. Such a sample may be a biological sample or may be a sample of an artificially generated aqueous solution. Such solutions, in an aspect, are obtained at different stages during neurotoxin manufacture, either for quality control and/or activity determination/specification purposes or for safety control. It is envisaged that the neurotoxin present in the said sample shall exhibit at least the neurotoxin protease activity. In another aspect, the neurotoxin is fully biologically active. In an aspect the said fully biologically active neurotoxin is required for entering the cell and for activating the read out based on the fusion polypeptide of the present invention. Accordingly, such a fully biologically active neurotoxin is to be applied if a host cell is to be contacted with the sample to be analyzed by the method of the invention. In another aspect, the sample to be applied for the method of the invention comprises neurotoxin polypeptides or fragments thereof which merely exhibit neurotoxin protease activity. Such neurotoxin polypeptides or fragments are, in an aspect, muteins of neurotoxin polypeptides comprising or consisting essentially of a proteolytically active light chain.

In an aspect, the reporter gene activity is determined by measuring the enzymatic conversion of a reporter gene substrate. The latter one can be measured in an aspect by detecting the intensity of the light emitted during the conversion reaction. Suitable systems for measuring the light emission that occurs during the conversion reaction catalyzed by reporter genes are well known in the art and commercially available. Moreover, suitable substrates which can be used for the reporter genes are also well known and commercially available.

The neurotoxin polypeptide in a sample can be determined quantitatively or qualitatively. For a qualitative determination, in an aspect of the invention, the presence or absence of a neurotoxin polypeptide is determined. For a quantitative detection, in an aspect, the amount of the neurotoxin polypeptide is determined. In an aspect, the quantitative determination encompasses a determination of the absolute amount or a relative amount, i.e. an amount which is normalized such that the amount found in different samples can be compared with each other. In an aspect, this can be achieved by comparison of a measured reporter gene activity for a test sample to a calibration curve which is to be established by subjecting calibration samples having predetermined amounts of the neurotoxin polypeptide to the method of the present invention.

In one aspect of the method of the invention, the host cell is a host cell as defined elsewhere herein.

The invention further relates to the use of the polynucleotide, the vector, the fusion polypeptide or the host cell of the invention for determining neurotoxin activity in a sample.

Finally, the invention pertains to a kit for determining neurotoxin activity comprising the polynucleotide, the vector, the fusion polypeptide or the host cell of the invention and, preferably, a detection agent for detecting reporter gene activity.

The term “kit” as used herein refers to a collection of means comprising the fusion polypeptide, the polynucleotide, the vector and/or the host cell of the present invention which are provided in separate or common vials in a ready to use manner for carrying out the method of the present invention. In an aspect, the kit comprises additional means for carrying out the method of the present invention, in an aspect, calibration standard solutions comprising neurotoxin polypeptide with defined biological activity and/or means for measuring the reporter gene activity such as detection agents for the reporter gene or substrates converted by the reporter gene. Furthermore, in an aspect, the kit comprises instructions for carrying out the method of the present invention. These instructions can be provided as a manual or can be in the form of a computer-implementable algorithm on a data storage medium which upon implementation is capable of governing one or more steps of the method of the invention. In an aspect, the kit is to be used for carrying out the method of the invention specified above.

All references cited in the specification are herewith incorporated by reference with respect to the entire disclosure content and the disclosure contents specifically mentioned in the specification.

### FIGURES

FIG. 1 A) shows the original-regulator plasmid provided by Clontech, which was used as basis for the construction of activator-constructs; B) Luciferase-encoding Reporter-construct; C) System control plasmid for Luciferase-reporter construct (all: Clontech).

FIG. 2 shows one example for the construction of the activator-plasmid mST01 (SEQ ID NO: 9) with pCMV-Tet3G-Vector (FIG. 1A) as backbone. The claimed polynucleotide comprising the N-terminal membrane anchor domain of Synaptotagmin (87 amino acids) and the 112 C-terminal amino acids of SNAP25 is cloned via the plotted EcoRI and XbaI restriction-sites. The key-features of the vector are highlighted as arrows and denoted in the table on the right indicating their position (in bp).

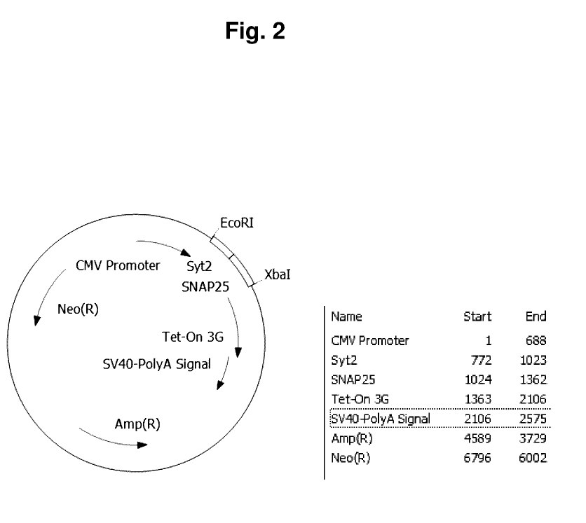

FIG. 3 shows the results of a genomic PCR as control for stable integration of the Activator- and Reporter construct. Primer designed for Luciferase yield a product of ˜1 kb, those for Tet-Element ˜0.5 kb. The highlighted lanes represent clones that stably integrated both plasmids. In total, 10 out of 24 generated clones were positive for Luciferase and Tet-Element, based on the results of the genomic PCR.

FIG. 4 shows a cartoon of the mode of action of the depicted example showing a membrane-anchored construct of Synaptotagmin-SNAP25 fusion protein.

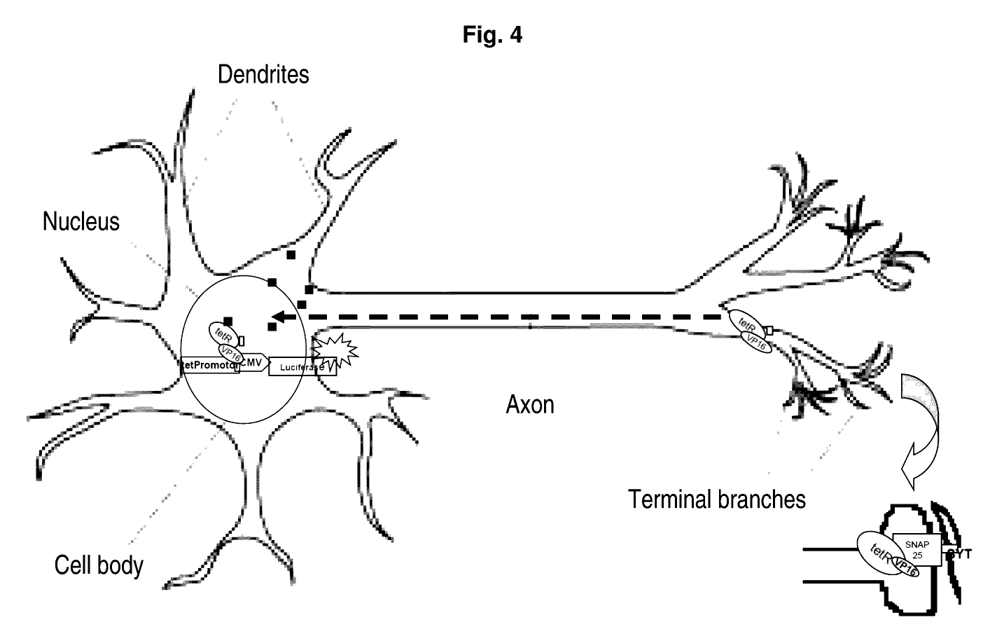

### ABBREVIATIONS AND SYMBOLS

SYT: Synaptotagmin;
tetR: Tetracycline-Repressor;
VP16: transcriptional activation domain of Herpes simplex VP16 protein;
CMV: Cytomegalie virus Promoter;
Black squares: Doxycycline.

The fusion protein is constitutively expressed and preferably linked to the membrane of terminal branches of neuronal differentiated cells. Upon cleavage of BoNT within the respective linker domain, the complex consisting of TetR-Element and VP16 is transported into the nucleus. In presence of the antibiotic doxycycline, a conformational change is induced which allows for the binding of the TetR-Element/VP16 to the Tet-Promoter and its thereby regulated reporter gene luciferase. Upon induced expression of luciferase, a detection and quantification of its enzymatic activity using commercially available assay kits is the functional readout. It is also applicable for all membrane-anchored constructs. For cytosolic activator constructs, localization is not defined to a preferred domain within the cell. The fundamental mechanism of transport of the TetR-Element into the nucleus and activation of the subsequent expression/detection cascade of the reporter is consistent.

### EXAMPLE

The invention will now be described by the following, non-limiting example which merely illustrates the invention but shall not be construed as limiting its scope.

A sub-cloned SiMa cell line (SIMA no. 3B4), derived from parental SiMa cell line (DSMZ, Acc.No.: ACC-164) by subcloning using limited dilution, has been stably transfected using Xfect Transfection Reagent and accompanied products provided by vendor (Clontech Laboratories, Inc., Cat. No.: 631317). Transfection was performed in 6-well plates, at the time of transfection cells showed 90% confluency. The respective polynucleotide-DNA was used at 2 μg per well. In case of transfection with the Luciferase-reporter-construct, said DNA was accompanied by 0.1 μg linear selection marker (ratio 20:1) for Hygromycin. After 4 h, medium containing transfection matrix was replaced by normal growth medium.

24 h after this medium change, two 6-wells of the same preparation have been pooled and plated onto PDL-coated Petri-dishes (014.5 cm) and let grown for at least 48 h (until density reached 70-80% confluence), before antibiotic selection (G418: 400 μg/ml; Hygromycin: 150 μg/ml) was applied until single colonies were formed. Those single colonies have been picked, expanded and subjected to testing for stable genomic integration of the respective construct of interest. After selection period antibiotic concentrations have been lowered to G418: 200 μg/ml and Hygromycin: 100 μg/ml.

Genomic PCR (cells were lysed using Lyse and Go PCR reagent, Thermo Scientific according to protocol) using specific primers for the Tet-Element (forward: AGCTGGGAGTTGAGCAGCCTAC (SEQ ID NO: 35); reverse: GGTGCCGAGATGCACTTTAGCC (SEQ ID NO: 36)) and Luciferase-Reporter (forward: GAAGAGATACGCCCTGGTTC (SEQ ID NO: 37); reverse: CGGGAGGTAGATGAGATGTG (SEQ ID NO: 38)) applying Phusion High Fidelity Kit (NEB) according to protocol and analysis by subsequent agarose gel electrophoresis.

After having been tested positively for the insertion of both constructs, cells have been expanded and cell bank stocks in liquid nitrogen tanks have been generated. Tests for stable insertion after re-thawing of stocks have been performed before using cells for experiments.

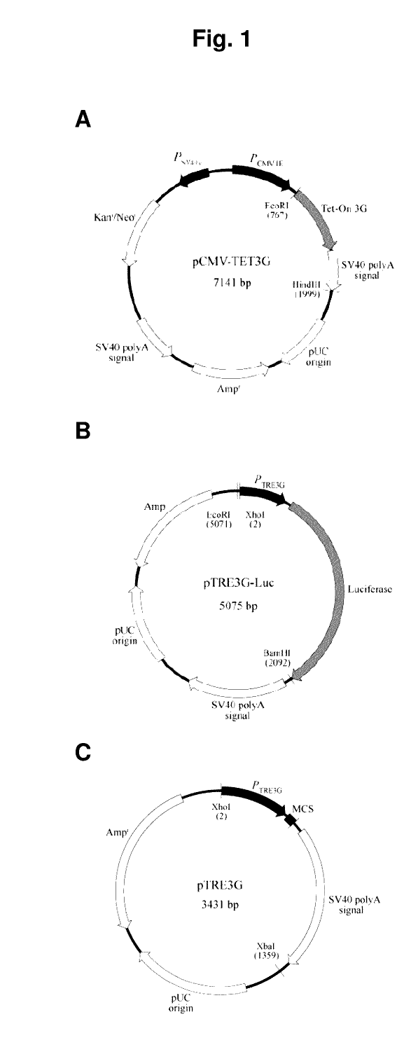

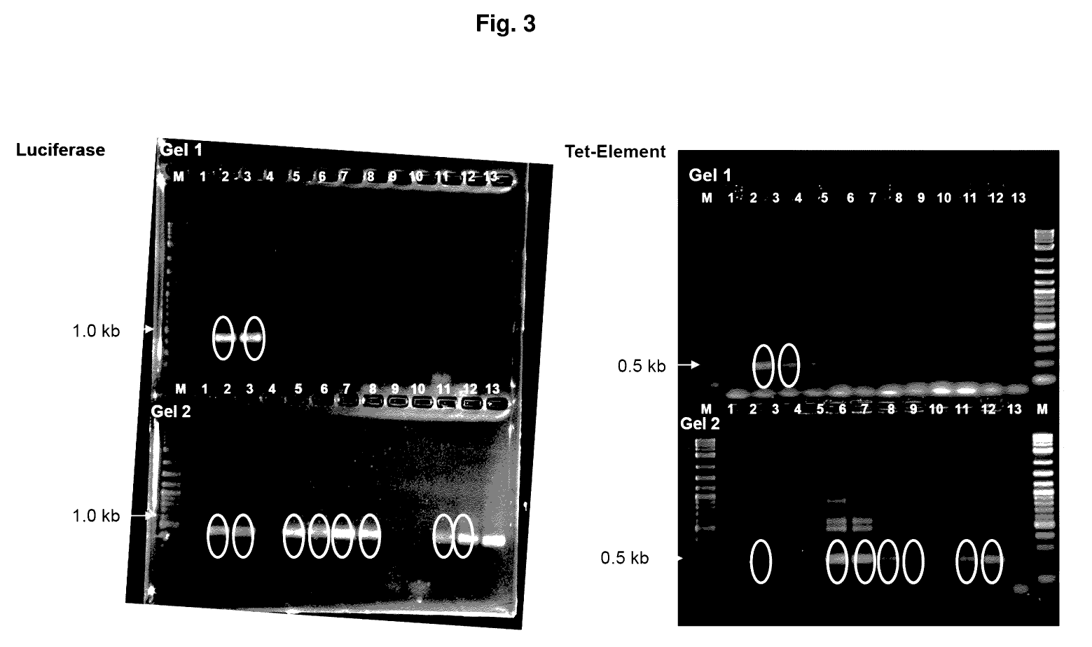

In the first place, construct mST01 (Synaptotagmin-SNAP-25-tTA) (SEQ ID NO: 9) was transfected and single cell subcloning was performed to reach a clonal background with as little genetic variation as possible. These stable clones were subjected to a second round of transfection with the reporter gene construct pTRE-Luc (luciferase reporter) and repeated subsequent single cell cloning (FIG. 3). Transfectional, viability and functional controls were made by using the empty vectors (pCMV-Tet3G as activator control plasmid and pTRE3G as reporter control plasmid) without the respective GOI and did not show any impairment in the above mentioned features (FIG. 1A, C) (Clontech).

After showing the stability of integration, the expression analysis of Tet-Element and cleavage of the BoNT linker-domain by neurotoxin is assessed. For this experiment, double-transfected clones are plated onto PDL-coated 96-well plates and differentiated to a neuronal phenotype according to the procedures disclosed in WO 2010/105234. Intoxication with BoNT/A is carried out using a concentration series with final concentrations in cell culture ranging from 1×10⁻¹⁰ M to 10×10⁻¹⁴M for 24-72 hours. The stock dilution series of BoNT is performed in DPBS supplemented with 0.2% human serum albumin and applied correspondingly and directly into the cell culture medium. After intoxication, the release of Tet-Repressor protein will be detectable and distinguishable from the original polypeptide sequence. Further on the expression level of the Luciferase-reporter is assessed in time and dose-response to BoNT as well as in a time and dose response level to doxycycline as a Tet-on System is used as basis. Functional readout is done by using commercially available Luciferase-Assay-Kits. In this case, two different kits can be applied, mainly for a purely functional read out a pure Luciferase Assay System (One-Glo, Promega) and for full assay application a Luciferase Assay System, in combination with assessment of cell viability (One-Glo+Tox, Promega). This multiplexing approach allows for the normalization and functional data within the same samples and provides, therefore, for a solid basis for validation of the assay.

1-17. (canceled)

18. A polynucleotide encoding a fusion polypeptide comprising (i) transcription factor domain and (ii) a cytoplasmic retention domain, separated by a linker comprising a neurotoxin cleavage site.

19. The polynucleotide of claim 18, wherein the cytoplasmic retention domain is a transmembrane protein or transmembrane spanning domain of a transmembrane protein.

20. The polynucleotide of claim 19, wherein the transmembrane protein is selected from the group consisting of plasmalemmal neurotransmitter transporters, ion channels, G-protein coupled receptors and membrane receptors.

21. The polynucleotide of claim 18, wherein the cytoplasmic retention domain is a membrane-anchored protein or a membrane anchor domain of a membrane-anchored protein.)

22. The polynucleotide of claim 21, wherein the membrane-anchored protein is selected from the group consisting of SNAP-25, Syntaxin, synaptobrevin, synaptotagmin, vesicle associated membrane proteins (VAMPs), synaptic vesicle glycoproteins (SV2), high affinity choline transporters, Neurexins, voltage-gated calcium channels acetylcholinesterase and NOTCH.

23. The polynucleotide of claim 18, wherein the cytoplasmic retention domain is selected from the group consisting of a globular protein and a cytoskeleton anchor protein.

24. The polyrrucleotide of claim 18, wherein the transcription factor domain comprises a transcription factor, wherein the transcription factor comprises a DNA binding domain, and a transactivator domain or a silencer domain.

25. The polynucleotide of claim 24, wherein the transcription, factor is selected from the group consisting of a tetracycline dependent transactivator (tet-repressor—VP 16), a steroid hormone receptor, NOTCH. NFκB, p53, NEAT, MLL, E2A, HSF1, NF-IL6, STAT, R-SMAD, GAL4, and SP1,

26. A vector comprising a polynucleotide of claim 18.

27. A fusion polypeptide encoded by the polynuceotide of claim 18.

28. A host cell comprising the polynucleotide of claim 18.

29. The host cell of claim 28, wherein the host cell is selected from the group consisting of primary neuronal cells, neuroblastoma cell lines, cell line N1E-115, cell line Neuro2a, cell line SH-SY5Y, cell line PC12, cell line SiMa, cell line MHH-NB-11, cell line SK-N-BE(2) and induced pluripotent stein cells.

30. The host cell of claim 28, wherein the host cell comprises a reporter gene, the expression of which is modulated by the transcription factor domain upon cleavage of the fusion polypeptide.

31. A host cell comprising the fusion polypeptide of claim 27.

32. A method for determining neurotoxin activity in a sample comprising the steps of:
(a) contacting a host cell comprising the fusion polypeptide of claim 27 and a reporter gene the expression of which is modulated by the transcription factor domain upon cleavage of the fusion polypeptide with a sample suspected to comprise neurotoxin activity; and
(b) measuring the reporter gene activity, whereby neurotoxin activity in the sample is determined.

33. The method of claim 32, wherein the host cell is selected from the group consisting of primary neuronal cells, neuroblastoma cell lines, cell line N1E-115, cell line Neuro2a, cell line SH-SY5SY, cell line PC12, cell line SiMa, cell line MHH-NB-11, cell line SK-N-BE(2) and induced pluripotent stem cells.

34. A kit for determining neurotoxin activity comprising the polynucleotide of claim 18 and a detection agent for detecting reporter gene activity.
